(19) United States
(12) Patent Application Publication
(10) Pub. No.: US 2024/0293176 A1
(43) Pub. Date: Sep. 5, 2024

(54) METHODS, DEVICES AND SYSTEMS FOR TREATING A PATIENT BY GSN ABLATION

(71) Applicant: Axon Therapies, Inc., New York, NY (US)

(72) Inventors: Manuel Arzadon JAVIER, JR., Santa Clara, CA (US); Pajhand IRANITALAB, San Ramon, CA (US)

(21) Appl. No.: 18/568,206

(22) PCT Filed: Jun. 6, 2022
(86) PCT No.: PCT/US2022/032391

§ 371 Date: Dec. 7, 2023

Related U.S. Application Data
(60) Provisional application No. 63/197,953, filed on Jun. 7, 2021.

(51) Int. Cl. A61B 18/14; A61B 18/00

(52) U.S. Cl. CPC A61B 18/1492; A61B 2018/00029; A61B 2018/00077; A61B 2018/00238; A61B 2018/00434; A61B 2018/00577; A61B 2018/00773; A61B 2018/00791; A61B 2018/1435; A61B 2218/002

(57) ABSTRACT

Systems, devices, and methods for transvascular ablation of target tissue. The devices and methods may, in some examples, be used for splanchnic nerve ablation to increase splanchnic venous blood capacitance to treat at least one of heart failure and hypertension. For example, the devices disclosed herein may be advanced endovascularly to a target vessel in the region of a thoracic splanchnic nerve (TSN), such as a greater splanchnic nerve (GSN) or a TSN nerve root. Also disclosed are methods of treating heart failure, such as HFpEF, by endovascularly ablating a thoracic splanchnic nerve to increase venous capacitance and reduce pulmonary blood pressure.

### INCORPORATION BY REFERENCE

This application claims priority to U.S. Provisional Application No. 63/197,953, filed Jun. 7, 2021, the disclosure of which is incorporated by reference herein in its entirety for all purposes.

All publications and patent applications mentioned in this specification are herein incorporated by reference to the same extent as if each individual publication or patent application was specifically and individually indicated to be incorporated by reference.

This disclosure is related by subject matter to U.S. Pub. Nos. US2019/0175912, US2019/0183569, US2021/0220043, U.S. Pat. Nos. 10,376,308, 10,207,110, application Ser. Nos. 16/510,503, 62/836,720, 62/837,090, 62/864,093, PCT/US2019/15400, PCT/US2020/038934, PCT/US2021/014001, and PCT Pub. Nos. WO2018/023132, WO2019/118976, and WO/2020/257763, all of which are incorporated herein by reference in their entirety for all purposes.

### BACKGROUND

Heart failure (HF) is a medical condition that occurs when the heart is unable to pump sufficiently to sustain the organs of the body. Heart failure is a serious condition and affects millions of patients in the United States and around the world.

One common measure of heart health is left ventricular ejection fraction (LVEF) or ejection fraction. By definition, the volume of blood within a ventricle immediately before a contraction is known as the end-diastolic volume (EDV). Likewise, the volume of blood left in a ventricle at the end of contraction is end-systolic volume (ESV). The difference between EDV and ESV is stroke volume (SV). SV describes the volume of blood ejected from the right and left ventricles with each heartbeat. Ejection fraction (EF) is the fraction of the EDV that is ejected with each beat; that is, it is SV divided by EDV. Cardiac output (CO) is defined as the volume of blood pumped per minute by each ventricle of the heart. CO is equal to SV times the heart rate (HR).

Cardiomyopathy, in which the heart muscle becomes weakened, stretched, or exhibits other structural problems, can be further categorized into systolic and diastolic dysfunction based on ventricular ejection fraction.

While a number of drug therapies successfully target systolic dysfunction and HFrEF, for the large group of patients with diastolic dysfunction and HFpEF no promising therapies have yet been identified. The clinical course for patients with both HFrEF and HFpEF is significant for recurrent presentations of acute decompensated heart failure (ADHF) with symptoms of dyspnea, decreased exercise capacity, peripheral edema, etc. Recurrent admissions for ADHF utilize a large part of current health care resources and could continue to generate enormous costs.

While the pathophysiology of HF is becoming increasingly better understood, modern medicine has, thus far, failed to develop new therapies for chronic management of HF or recurrent ADHF episodes. Over the past few decades, strategies of ADHF management and prevention have and continue to focus on the classical paradigm that salt and fluid retention is the cause of intravascular fluid expansion and cardiac decompensation.

Thus, there remains a need for improved therapies for heart failure patients that are safe and effective, and devices and systems that are adapted and configured to perform those therapies. A need also remains for safely and effectively delivering medical instruments to desired anatomical locations so those instruments can be used to perform those therapies.

### SUMMARY OF THE DISCLOSURE

The disclosure is related to methods of, devices for, and approaches for ablating a thoracic splanchnic nerve or a thoracic splanchnic nerve root. The ablations can be performed to treat at least one of hypertension and heart failure, but the general methods may also be used for other treatments as well. For example, the methods herein can be used in the treatment of pain, or even to generally benefit the subject to reducing the amount of blood that is expelled from the splanchnic bed into the central thoracic veins.

The treatments herein may be accomplished by increasing splanchnic capacitance. The therapies generally include ablating a patient's preganglionic thoracic splanchnic nerve or thoracic splanchnic nerve root to increase splanchnic capacitance, and thereby treat at least one of hypertension and heart failure.

Methods herein describe ablating thoracic splanchnic nerves, such as a greater splanchnic nerve or greater splanchnic nerve roots. While methods herein may provide specific examples of targeting greater splanchnic nerve or greater splanchnic nerve roots, it may be possible to alternatively, or in addition to, ablate other thoracic splanchnic nerves (e.g., lesser, least) to perform one or more treatments herein.

One aspect of the disclosure is a catheter delivery system (e.g., 500) comprising a delivery sheath, a first dilator (e.g., 530) and a second dilator (e.g., 550). Any of the features described with respect to this aspect may be combined with any other suitably combinable feature in this aspect.

In this aspect, the first and second dilators may each comprise a dilator distal section (e.g., 541, 551) that extends from a distal end (e.g., 508) of the delivery sheath when fully inserted by an extension amount in a range of 10 cm to 30 cm. Each of the dilator distal sections may comprise a stiffness that is less than a stiffness of a distal section (e.g., 514) of the delivery sheath. A stiffness of a second dilator distal section may be less than a stiffness of the first dilator distal section. A stiffness of a dilator distal sections may decrease in a distal direction. Dilator distal sections may comprise a distally decreasing outer diameter. A distally decreasing outer diameter may comprise a gradual taper, with stepped down outer diameters, or with a combination thereof.

In this aspect, the first dilator and the second dilator may each comprise a dilator tubular structure, a proximal end, a distal end, a working length between the proximal and distal ends, a central lumen therebetween, a distal section with a distally decreasing outer diameter, and a tapered distal tip.

In this aspect, wherein each of the dilator working lengths (e.g., 536, 556) may be in a range of 60 cm to 145 cm.

In this aspect, the first dilator's distal section (e.g., 541) may have a length in a range of 3 to 10 cm, preferably 5+/−0.5 cm.

In this aspect, the second dilator's distal section (e.g., 551) may have a length in a range of 3 to 10 cm, preferably 5+/−0.5 cm.

In this aspect, the second dilator may have a preformed curve (e.g., 552) on a distal section (e.g., 551). A preformed curve, when in an unconstrained state, may comprise an angle (e.g., 553) in a range of 90 degrees to 120 degrees, optionally 115 degrees, a radius of curvature 554 in a range of 7 to 11 mm, and optionally 9.14 mm. A second dilator may comprise a straight section (e.g., 555) distal to a preformed curve with a length in a range of 5 mm to 10 mm, preferably 7 mm. A tapered distal tip of each dilator may have a length in a range of 3 to 10 mm, preferably 5+/−0.5 mm.

In this aspect, the system may further comprise a guidewire.

In this aspect, the delivery sheath may comprise a proximal end and a distal end, a lumen therebetween, and a tubular structure (e.g., 506) comprising a braided wire and a polymer. A tubular structure may have a variable stiffness that decreases towards the distal end. A variable stiffness may change on a graduation. A variable stiffness may change in sections. A variable stiffness may be created by varying a braid density of the braided wire. A braid density proximate the proximal end may be 80 PPI, and a braid density proximate the distal end may be 40 PPI.

In this aspect, a tubular structure may comprise a proximal section comprising a first stiffness, a middle section comprising a second stiffness, and a distal section comprising a third stiffness, wherein the third stiffness is less than the first stiffness and the second stiffness is between that of the first and third stiffnesses. A proximal section may comprise a braid density of 80 PPI and a polymer with a durometer of 72D, the middle section may comprise a braid density of 60 PPI and a polymer with a durometer of 63D, and the distal section may comprise a braid density of 40 PPI and a polymer with a durometer of 55D. Proximal, middle, and distal sections may each comprise an inner diameter equal to one another, optionally 3.35 mm. Proximal, middle, and distal sections may each comprise a wall thickness equal to one another, optionally 0.127 mm.

In this aspect, the system may be configured for delivering an ablation catheter from a vasculature access point to the patient's azygos vein at a level between T7 and T11.

In this aspect, the system may be configured for delivering an ablation catheter from a vasculature access point to the patient's intercostal vein a level between T7 and T11.

In this aspect, a working length (e.g., 515) of the tubular structure of the sheath may be in a range of 50 cm to 115 cm, and optionally from 50 cm to 85 cm if an access point is a jugular vein, and optionally from 70 cm to 115 cm if an access point is a femoral vein. A distal section of the sheath may have a length of 9.50+/−0.50 cm. A middle section of the sheath may have a length of 6.5+/−0.5 cm. A proximal section of the sheath may have length that is the remainder of the working length minus a length of the distal section and a length of the middle section. A proximal section of the sheath may have a length of 64 cm.

In this aspect, the delivery sheath may be, or include any of the features of, any of the delivery sheaths described, claimed or shown herein.

In this aspect, the catheter delivery system may be provided as a kit in a sterilized package.

One aspect of the disclosure is a method of using a delivery system, comprising: advancing a delivery sheath within a patient: advancing a dilator from with the delivery sheath beyond a distal end of the delivery sheath: advancing the dilator into an azygos vein from a vena cava; and further advancing the delivery sheath over the dilator and into the azygos vein. In this aspect, advancing the dilator beyond a distal end of the delivery sheath comprises advancing the dilator from 10 cm to 30 cm beyond a distal end of the delivery sheath.

One aspect of the disclosure is a method of ablating a splanchnic nerve of a patient, comprising: delivering a delivery sheath to an azygos vein: delivering an ablation catheter comprising one or more ablation elements through the delivery sheath and positioning the one or more ablation elements proximate to tissue: measuring and storing a baseline central venous pressure (CVP_{b}): delivering ablation energy from an ablation console to the one or more ablation elements and to the tissue: measuring a second central venous pressure (CVP₂); and comparing the CVP₂ to the CVP_{b}. Any of the features described with respect to this aspect may be combined with any other suitably combinable feature in this aspect.

In this aspect, the delivery sheath may comprise a pressure sensor, and the step of measuring and storing a CVP_{b} may comprise obtaining a signal from the pressure sensor.

In this aspect, measuring and storing the CVP_{b} may be performed by a processor in the ablation console.

In this aspect, measuring and storing the CVP_{b} may be performed within a predefined period before delivering the ablation energy, such as within 10 minutes, within 5 minutes, or within 1 minute.

In this aspect, measuring the CVP₂ may be done while delivering ablation energy and/or following completion of delivering the ablation energy.

In this aspect, delivering a delivery sheath to an azygos vein may comprise introducing the delivery sheath into a femoral vein.

In this aspect, delivering a delivery sheath to an azygos vein may comprise introducing the delivery sheath into a jugular vein.

In this aspect, delivering a delivery sheath to an azygos vein may comprise delivering the delivery sheath to a region in the azygos vein between a T7 level and a T11 level.

In this aspect, positioning the one or more ablation elements may comprise positioning the one or more ablation elements within a region in an intercostal vein between an ostium from the azygos vein and 25 mm from the ostium.

In this aspect, the method may further include performing a success step if the CVP 2 is less than or equal to the CVP_{b} minus a predefined pressure, wherein optionally the predefined pressure is at least one of 10 mmHg, or more than 20 mmHg, or a user defined value.

In this aspect, a success step may comprise delivering a user message on a user interface on the ablation console. A user message may comprise the difference between the CVP b and the CVP₂. A user message may comprise the predefined pressure. If the CVP₂ comprises a plurality of measurements taken over time, a user message may comprise a visual representation (optionally a graph) showing the CVP_{b} and the CVP₂ on a graph with time, and optionally a graph shows the predefined pressure.

In this aspect, the measuring steps may occur or take place in the patient's vena cava.

In this aspect, the delivery sheath may be any of the delivery sheaths herein.

In this aspect, the CVP₂ may comprise a plurality of measurements obtained over time.

One aspect of this disclosure is a pressure monitoring delivery sheath (e.g., 480) comprising a proximal end (e.g., 486), a distal end (e.g., 487), a tubular section (e.g., 481) comprising a wall and a lumen (e.g., 491), and one or more pressure sensors (e.g., 483). Any of the features described with respect to this aspect may be combined with any other suitably combinable feature in this aspect.

In this aspect, the one or more pressure sensors may be positioned in a range that starts from 24 cm to 48 cm from the distal end.

In this aspect, the one or more pressure sensors may be positioned in a range that starts from 32 cm to 56 cm from the proximal end of the tubular section.

In this aspect, the tubular section may have a working length (e.g., 482) in a range of 50 cm to 115 cm.

In this aspect, the one or more pressure sensors may be positioned in the wall of the tubular section.

In this aspect, the one or more pressure sensors may comprise one or more of an optical sensor, a strain sensor, a film sensor, or a variable capacitance sensor.

In this aspect, the one or more pressure sensors may comprise a MEMS sensor.

In this aspect, the one or more pressure sensors may comprise a plurality of pressure sensors positioned at one or both of different radial positions or different axial positions of the tubular section.

In this aspect, the one or more pressure sensors may be covered in a protective pressure-transmitting cover, which may optionally be a flexible membrane, and which may optionally be flush with an outer surface of the tubular structure.

In this aspect, the one or more pressure sensors may be electrically connected to a connector, and the connector may be configured and adapted to be connectable to a pressure measuring console.

In this aspect, the tubular section may have a working length that allows a distal region of the tubular section to reach an azygos vein between a T7 level and a T11 level from an access point, wherein the access point may be a femoral vein or a jugular vein. A working length of the tubular section may be in a range of 50 cm to 115 cm, optionally 80 cm.

In this aspect, the sheath may further comprise a deployable balloon (e.g., 583, 603) proximate the distal end of the tubular section that may be disposed on an outer surface of the tubular section.

In this aspect, a deployable balloon may be positioned and adapted to be deployed radially asymmetric about the delivery sheath and positioned and adapted to deploy on a first radial side of the delivery sheath. A return electrode may be disposed at least on a side opposite the first radial side. A return electrode may be within 15 cm of the distal end of the tubular section. A return electrode may have a surface area in a range of 10 mm² to 200 mm². A return electrode may comprise a plurality of electrodes each having a length in a range of 1 mm to 10 mm and spaced from one another with a space in a range of 5 to 10 mm. A return electrode may comprise radiopaque material. A temperature sensor may be positioned on a side opposite the first radial side.

In this aspect, the sheath may further comprise a contrast delivery lumen in a wall of the tubular section, the contrast delivery lumen in fluid communication with a port positioned proximate the distal end of the tubular section. A port may comprise a pressure release valve, optionally wherein the pressure release valve is rated to open when a pressure in the contrast delivery lumen is greater than exterior to the valve by a range of 50 to 150 mmHg.

One aspect of the disclosure is a pressure monitoring delivery sheath, comprising: a tubular section comprising a proximal end, a distal end, a wall, a lumen, and a pressure sensor that is positioned in a range that starts from 24 cm to 48 cm from the distal end and starts from 32 cm to 56 cm from the proximal end of the tubular section, the tubular section having a working length in a range of 50 cm to 115 cm. In this aspect, the sheath may include any feature of the any pressure monitoring delivery sheath herein.

One aspect of this disclosure is a method of ablating a splanchnic nerve of a patient, comprising: delivering a delivery sheath into an azygos vein; delivering inflation fluid through an inflation lumen in the sheath to inflate an inflatable structure proximal a distal end of the delivery sheath: delivering an ablation catheter comprising one or more ablation elements through the delivery sheath and positioning the one or more ablation elements proximate to tissue; and delivering ablation energy to the one or more ablation elements and to the tissue. In this aspect, inflating the inflatable structure may reduce blood flow in the azygos vein.

One aspect of this disclosure is a delivery sheath comprising a proximal end, a distal end, a lumen therebetween, and a deployable balloon (e.g., 583) proximate the distal end and on an outer surface. In this aspect, the delivery sheath may include any other suitably combinable feature of any of the sheaths herein.

One aspect of the disclosure is a delivery sheath, comprising: a proximal end, a distal end, a lumen therebetween, and a deployable balloon proximate the distal end and on an outer surface of the delivery sheath, the deployable balloon positioned and adapted to be deployed radially asymmetric about the delivery sheath and positioned to be deployed on a first radial side of the delivery sheath. In this aspect, the delivery sheath may include any other suitably combinable feature of any of the sheaths (e.g., delivery sheaths) herein.

One aspect of this disclosure is a delivery sheath, comprising a proximal end, a distal end, a lumen defined by a wall therebetween, a contrast delivery lumen in the wall, the contrast delivery lumen in fluid communication with a port positioned proximate the distal end. The port optionally comprises a pressure release valve, optionally rated to open when a pressure in the contrast delivery lumen is greater than exterior to the valve by a range of 50 to 150 mmHg. In this aspect, the sheath may further comprise a contrast delivery connector and a stop cock valve.

### BRIEF DESCRIPTION OF THE DRAWINGS

The drawings included herewith are for illustrating various examples of articles, methods, and apparatuses of the present specification and are not intended to limit the scope of what is taught in any way. In the drawings:

FIG. 1 is an isometric view schematic illustration of an ablation catheter positioned in an intercostal vein for ablation of a thoracic splanchnic nerve.

FIG. 2 is a transverse view schematic illustration of an ablation catheter positioned in an intercostal vein and a centered azygos vein.

FIG. 3 is an AP fluoroscopic image of a patient's T8 to T12 thoracic region.

FIG. 4 is an RAO30 fluoroscopic image of a patient's T8 to T12 thoracic region.

FIG. 5A is a schematic illustration of an ablation catheter with two coiled RF electrodes.

FIG. 5B is a schematic illustration of an ablation catheter with two coiled RF electrodes and a distal deployable element.

FIG. 5C is a schematic illustration of a first, second and third section of a catheter shaft.

FIG. 5D is a schematic illustration of a distal portion or section of an ablation catheter having irrigation holes arranged in a helical pattern between windings of a helical electrode and an irrigation hole distal to the distal electrode.

FIG. 5E is a schematic illustration of a distal portion of an ablation catheter having irrigation holes arranged in a helical pattern between at least some windings of a helical electrode and a plurality of irrigation holes distal to a distal electrode and between proximal and distal electrodes.

FIG. 6 is a schematic illustration of an ablation catheter with an RF electrode comprising an expandable balloon with an RF electrode on its surface.

FIGS. 7A and 7B are schematic illustrations of an ablation catheter with RF electrode pads on an expandable balloon.

FIG. 8 is a schematic illustration of an ablation catheter with ultrasound transducers.

FIG. 9 is a flowchart of a method of treatment.

FIG. 10 is a schematic illustration of an exemplary delivery sheath with a pressure monitoring sensor.

FIG. 11A is a schematic illustration of an exemplary delivery sheath with a deployable balloon.

FIG. 11B is a schematic illustration of an exemplary delivery sheath with an asymmetric deployable balloon and a return electrode.

FIGS. 12A to 12D are various views of components of an exemplary delivery system.

### DETAILED DESCRIPTION

The disclosure herein is generally related to methods of treating at least one of heart failure and hypertension by increasing splanchnic capacitance. Some approaches include systems, devices, and methods for transvascular (e.g., transvenous) ablation of target tissue to increase splanchnic venous capacitance or venous compliance. The devices and methods may, in some examples, be used for ablating a splanchnic nerve to increase splanchnic capacitance. For example, the exemplary ablation devices disclosed herein may be advanced endovascularly to a target vessel or plurality of vessels in the region of a thoracic splanchnic nerve (“TSN”), such as a preganglionic greater splanchnic nerve (“GSN”), lesser splanchnic nerve, or least splanchnic nerve or one of their roots (a TSN nerve root). The target vessel may be, for example, an intercostal vein or an azygos vein (or both) or a vein of the azygos vein system, preferably, one or more of the lowest (i.e., most caudal) three intercostal veins (which may be T9, T10, or T11).

Methods herein describe ablating thoracic splanchnic nerves, such as a greater splanchnic nerve or greater splanchnic nerve roots. While methods herein may provide specific examples of targeting greater splanchnic nerve or greater splanchnic nerve roots, it may be possible to alternatively, or in addition to, ablate other thoracic splanchnic nerves (e.g., lesser, least) to perform one or more treatments herein.

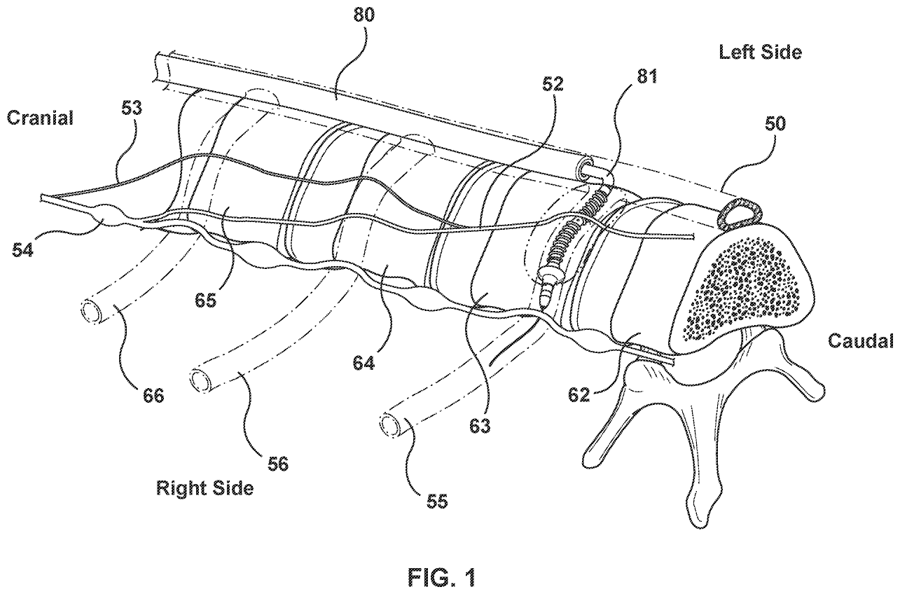

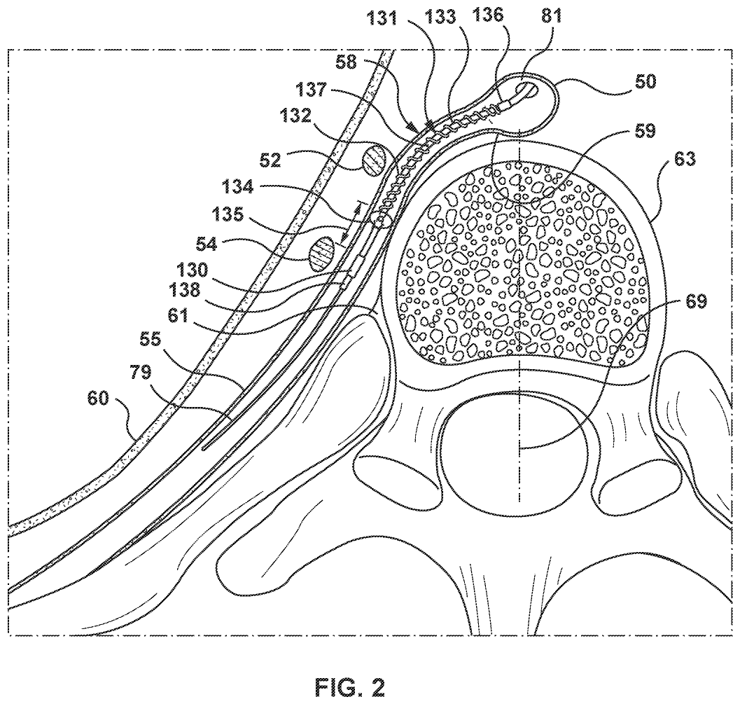

FIG. 1 illustrates a non-limiting exemplary location for placement of an exemplary ablation catheter. FIG. 1 shows a patient's thoracic spine, including T12 (62). T1l (63), T10 (64), and T9 (65) vertebrae, intervertebral discs, a sympathetic trunk 54, an azygos vein 50, a right T11 intercostal vein 55, a right T10 intercostal vein 56, a right T9 intercostal vein 66, GSN roots 53, and a fully-formed GSN 52. The lesser and least splanchnic nerves and their roots are omitted for simplicity. FIG. 1 illustrates an exemplary ablation catheter placement for ablating a GSN or its roots, additional examples of which are discussed herein. It is noted that ablation of the lesser or least splanchnic nerves or their roots may also have therapeutic effects and may be a procedural objective. An exemplary delivery sheath 80 (which may include any number of features of any of the delivery sheaths herein) is shown positioned in the azygos vein and an ablation catheter 81 is shown delivered through the sheath and passing from the azygos vein into the T1l intercostal vein. The sympathetic trunk runs substantially parallel to the spine, consistently passing close to each costovertebral joint 61 (see FIG. 2). On the right side of the body the GSN roots branch from the sympathetic trunk, typically cranial to the T9 vertebra, and converge to form the GSN, which travels at an angle from the sympathetic trunk toward the anterior-center of the spine and is positioned anterior to the intercostal veins between the intercostal veins and parietal pleura 60 (see FIG. 2). The azygos vein 50 travels along the anterior of the spine and may be somewhat straight and parallel to the axis of the spine as shown in FIG. 1.

An endovascular approach to transvascularly ablate a TSN, particularly a GSN may involve one or more of the following steps: accessing venous vasculature at the patient's jugular vein or femoral vein with an access introducer sheath (e.g. 12F); delivering a delivery sheath (e.g., 9F sheath) to an azygos vein (e.g., to one or two thoracic levels above the target intercostal vein); in some embodiments, delivering contrast agent through the sheath to show location of veins on fluoroscopy: in some embodiments, delivering a guidewire (e.g., 0.014″ guidewire) through the delivery sheath and into a targeted T9, T10, or T11 intercostal vein; and delivering an ablation catheter through the delivery sheath to the azygos vein, in some embodiments over the guidewire, positioning an ablation element in an intercostal vein, azygos vein or both; and optionally aligning a radiopaque marker on the ablation catheter with an anatomical landmark (or positioning it relative thereto) to position an ablation element in a region that maximizes efficacy of ablating a target TSN/GSN while minimizing risk of injuring one or more non-target structures.

Some important anatomical structures in the vicinity of this region that should not be injured include the sympathetic trunk 54, vagus nerve, thoracic duct, and esophagus. Therefore, to ensure safety an ablation zone should be contained within a safe region that does not injure such structures.

Bones, blood vessels if injected with radiopaque contrast medium, and medical devices if made from radiopaque material, are visible on fluoroscopy but nerves are not. An ablation device designed for transvascular (e.g., transvenous) ablation of a TSN (e.g., GSN) from an intercostal vein, azygos vein, or both along with procedural steps may be provided to ensure efficacious ablation of the TSN (e.g., GSN) while ensuring safety. The procedural steps may include fluoroscopic imaging to position the ablation element(s) of the ablation catheter with respect to boney or vascular structures.

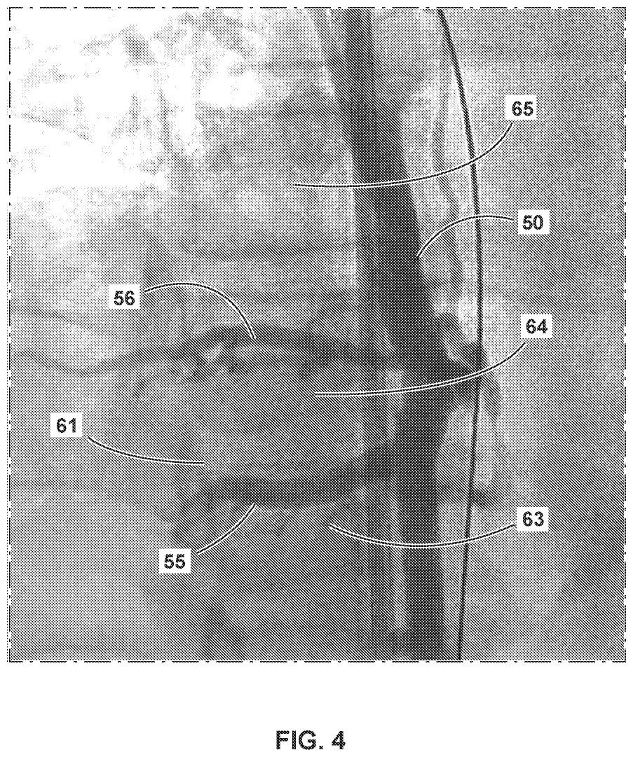

In a first embodiment of a method of ablating a right GSN a merely exemplary ablation catheter having a proximal radiopaque marker 136, a distal radiopaque marker 130, an ablation element 131 or plurality of ablation elements 132, 133, and an optional gap 135 between the ablation element and the distal radiopaque marker is advanced from an azygos vein 50 into an intercostal vein 55 at one of the lower three thoracic levels (e.g., T9, T10, T1l). The C-Arm is placed in Anterior-Posterior (AP) orientation. In some embodiments, the position of a distal radiopaque marker 130 relative to the costovertebral joint may be assessed (e.g., with the C-Arm in a RAO orientation) to ensure the sympathetic trunk is not at risk of injury The C-Arm may be obliquely angled to the right (RAO orientation) to maximize the 2D projection of the section of intercostal vein between the costovertebral joint 61 and anterior midline of the vertebra 69 (FIG. 4). For example, the C-arm may be positioned with a Right Anterior Oblique (RAO) angle in a range of 20° to 70° from AP (e.g., in a range of 30° to 60°, in a range of 35° to 55°, about 30°, at an angle that maximizes projected distance between the proximal and distal RO markers). With this view the user may check to make sure the distal radiopaque marker is not too close to the costovertebral joint 61. For example, if the distal radiopaque marker is positioned directly distal to the ablation element a distance of at least 3 mm (e.g., at least 5 mm) may be chosen to ensure the sympathetic trunk is not injured. In another example, if the distal radiopaque marker is positioned distal to the ablation element with a known space between them the distal radiopaque marker may be aligned with the costovertebral joint or proximal to it to ensure safety of the sympathetic joint. If the distal radiopaque marker is too close to or beyond the costovertebral joint the catheter may be pulled back until an acceptable distance between the distal radiopaque marker and the costovertebral joint is seen If the ablation element is comprised of a plurality of ablation elements (e.g., two) an ablation may first be performed from the more proximal ablation element prior to pulling the catheter back to appropriately place the distal radiopaque marker relative to the costovertebral joint. Then a subsequent ablation may be made from the more distal ablation element.

In a second embodiment of a method of ablating a right GSN an ablation catheter having a proximal radiopaque marker 136, a distal radiopaque marker 130, an ablation element 131 or plurality of ablation elements 132, 133, and an optional gap 135 between the ablation element and the distal radiopaque marker is advanced from an azygos vein 50 into an intercostal vein 55 at one of the lower three thoracic levels (e.g., T9, T10, T11). The C-Arm is placed in Anterior-Posterior (AP) orientation. The proximal radiopaque marker 136 may be aligned with the intercostal vein ostium 59 or at the midline of the vertebra 69. The ostium can be found for example by injecting contrast agent and viewing the vasculature on fluoroscopy or if a guidewire was previously positioned in a target intercostal vein a bend in the guidewire or ablation catheter may indicate the location of the ostium. In some embodiments, the position of a distal radiopaque marker 130 relative to the costovertebral joint may be assessed (e.g., with the C-Arm in a RAO orientation) to ensure the sympathetic trunk is not at risk of injury The C-Arm may be obliquely angled to the right (RAO orientation) to maximize the 2D projection of the section of intercostal vein between the costovertebral joint 61 and anterior midline of the vertebra 69 (FIG. 4). For example, the C-arm may be positioned with a Right Anterior Oblique (RAO) angle in a range of 20° to 70° from AP (e.g., in a range of 30° to 60°, in a range of 35° to 55°, about 30°, at an angle that maximizes projected distance between the proximal and distal RO markers). With this view the user may check to make sure the distal radiopaque marker is not too close to the costovertebral joint 61. For example, if the distal radiopaque marker is positioned directly distal to the ablation element a distance of at least 3 mm (e.g., at least 5 mm) may be chosen to ensure the sympathetic trunk is not injured. In another example, if the distal radiopaque marker is positioned distal to the ablation element with a known space between them the distal radiopaque marker may be aligned with the costovertebral joint or proximal to it to ensure safety of the sympathetic joint. If the distal radiopaque marker is too close to or beyond the costovertebral joint the catheter may be pulled back until an acceptable distance between the distal radiopaque marker and the costovertebral joint is seen, which may place the proximal radiopaque marker in the azygos vein especially if the azygos vein is right biased.

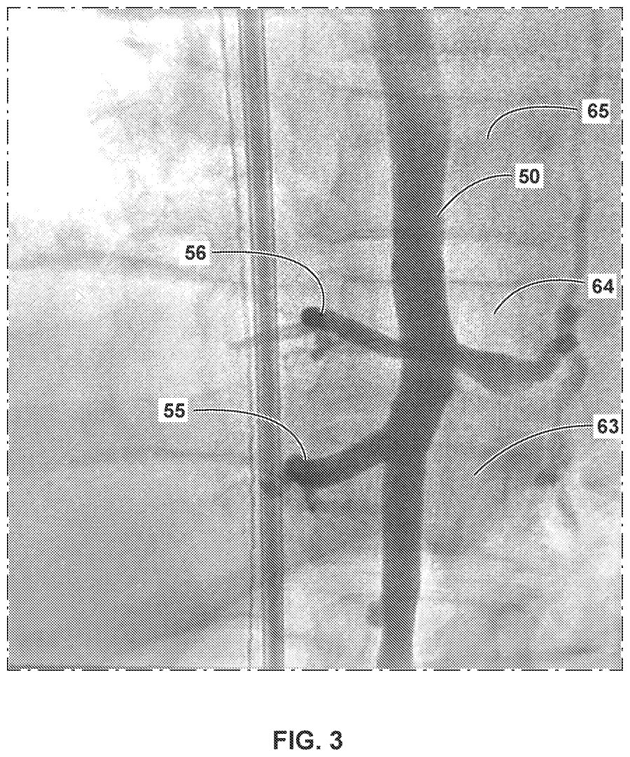

In a third embodiment of a method of ablating a right GSN an ablation catheter having a distal radiopaque marker 130, an ablation element 131 or plurality of ablation elements 132, 133, and a gap 135 between the ablation element and the distal radiopaque marker is advanced from an azygos vein 50 into an intercostal vein 55 at one of the lower three thoracic levels (e.g., T9, T10, T11). The C-Arm is obliquely angled to the right to maximize the 2D projection of the section of intercostal vein between the costovertebral joint 61 and anterior midline of the vertebra 69 (FIG. 2). For example, the C-arm may be positioned with a Right Anterior Oblique (RAO) angle in a range of 20° to 70° from AP (e.g., in a range of 30° to 60°, in a range of 35° to 55°, about 30°, at an angle that maximizes projected distance between the proximal and distal RO markers). A fluoroscopy image in an anterior-posterior (AP) view is shown in FIG. 3. In comparison a fluoroscopy image in a RAO 30° is shown in FIG. 4. The catheter may be advanced to align the distal radiopaque marker 130 with the costovertebral joint 61. Since the sympathetic trunk 54 is next to the costovertebral joint 61 the gap between the distal radiopaque marker and the ablation element may ensure the sympathetic trunk is not injured. The gap may be for example a length in a range of 0 to 25 mm (e.g., a range of 3 to 25 mm, a range of 5 to 25 mm, a range of 5 to 20 mm). In some embodiments, an inflatable balloon 134 may be positioned on the catheter shaft within the gap, which may help to anchor the catheter or contain ablation energy proximal to the balloon. In some embodiments, the catheter shaft 138 distal to the ablation element may be narrower or more flexible than the remainder of the shaft to facilitate delivery through the narrower distal portion of the intercostal vein. In some embodiments, the ablation element(s) has a length capable of ablating to the anterior midline of the vertebra 69 when the distal radiopaque marker is aligned with the costovertebral joint. For example, the ablation element(s) may have a total length in a range of 5 to 25 mm (e.g., in a range of 10 to 25 mm, in a range of 15 to 20 mm). The ablation catheter may have a proximal radiopaque marker located just proximal to the ablation element(s). In some embodiments, prior to delivering ablation energy a user may image the proximal radiopaque marker to ensure it is at the anterior midline of the vertebra 69. If the proximal radiopaque marker is to the left of the midline 69, for example if the patient is extremely small, there may be a risk of injuring a non-target tissue such as the thoracic duct or esophagus. To mitigate this risk a catheter with a smaller sized ablation element may be used or if the ablation element is made of a plurality of ablation elements only the elements between the midline 69 and distal radiopaque marker may be activated for ablation. Conversely, if the proximal radiopaque marker is to the right of the midline 69, for example if the patient is extremely large, there may be a risk of missing the GSN. To mitigate this risk another ablation may be performed at another intercostal level or within the same intercoastal vein with the position of the ablation element retracted until the proximal radiopaque marker is aligned with the midline 69.

In a fourth embodiment of a method of ablating a right GSN an ablation catheter having an ablation element 131, which may include a plurality of ablation elements, a distal radiopaque marker located at a distal end of the ablation element(s), and a proximal radiopaque marker located at a proximal end of the ablation element(s) is advanced from an azygos vein into an intercostal vein at one of the lower three thoracic levels (e.g., T9, T10, T11). The C-Arm is obliquely angled to the right to maximize the 2D projection of the section of intercostal vein between the costovertebral joint 61 and anterior midline of the vertebra 69 (FIG. 2). For example, the C-arm may be positioned with a Right Anterior Oblique (RAO) angle in a range of 25° to 65° from AP (e.g., in a range of 30° to 60°, in a range of 35° to 55°, about 30°). The catheter is advanced to align the distal radiopaque marker with a position relative to the costovertebral joint and the opposing edge of the vertebral body in the oblique view. For example, the distal radiopaque marker may be aligned with a point that is midway between the costovertebral joint and the opposing edge of the vertebral body in the oblique view. The ablation element(s) may have a total length expected to cover the GSN position range 68 in most patients. Ablation energy may be delivered from the ablation element(s) to ablate the range without moving the catheter. In some embodiments, the catheter may be moved to another intercostal level and a second ablation may be made using the same method steps.

Performing any of the exemplary embodiments of placement strategy disclosed above, when the ablation element 131 has a total length less than 30 mm (e.g., less than 25 mm, less than 20 mm, about 15 mm) it is expected that in a large majority of patients the sympathetic trunk will be spared from injury. Additionally, when performing the methods herein, when the ablation element 131 has a total length greater than or equal to 15 mm it is expected that in a large majority of patients the GSN will be ablated. Therefore, the exemplary ablation element 131 may have a total length in a range of 15 mm to 30 mm to be effective and safe for a large majority of patients using these placement strategies. However, smaller ablation element total length may be suitable for some patients. For example, the ablation element may have a total length in a range of 5 to 25 mm (e.g., in a range of 10 to 20 mm, or in a range of 10 to 15 mm).

As used herein, ablation element may refer to a single structure or a plurality of structures. For example, as used herein, ablation element may include a plurality of ablation electrodes that are axially spaced apart, and each of which may be adapted to facilitate the delivery of ablation energy.

Once acceptable ablation element placement is achieved, for example using one of the exemplary embodiments of placement strategy herein, ablation energy may be delivered from the ablation element or plurality of ablation elements without having to move the catheter. Ablation energy may be delivered from the ablation element to ablate tissue circumferentially around the intercostal vein a depth in a range of 2 mm to 10 mm (e.g., a range of 2 mm to 8 mm, a range of 3 mm to 8 mm, about 5 mm). In some embodiments, the procedure may be repeated at another thoracic level (e.g., a more cranial level, a more caudal level, another of T9, T10, T11 intercostal veins on the same side of the patient) especially if the azygos is right biased. Alternatively or in addition to having distal and proximal radiopaque markers at both ends of an ablation element or plurality of ablation elements, the ablation element(s) itself may be radiopaque and the same methods herein may be used to position the distal or proximal end of the ablation element(s) relative to anatomical landmarks (e.g., midline of the spine, costovertebral joint, etc.). The phrase radiopaque marker as used herein may thus describe an ablation element if the ablation element is radiopaque. In some alternative embodiments, a radiopaque markers may comprise a relatively longer radiopaque marker positioned under or next to one or more ablation elements wherein the proximal end of the long radiopaque marker is at least aligned with the proximal end of the ablation element or extending proximal of the ablation element by up to 3 mm and the distal end of the long radiopaque marker is at least aligned with the distal end of the ablation element or extending distal to the ablation element by up to 3 mm.

With any of the exemplary embodiments of placement strategy disclosed above, there may be situations when a portion of the ablation element(s) is in the azygos vein while the remainder is in the intercostal vein, in particular when the ablation catheter has an ablation element or plurality of elements having a total length in a range of 10 to 25 mm. The azygos vein is larger than the intercostal vein and has greater blood flow, which may impact the ability to create an effective ablation around the azygos vein or even in the intercostal vein and may require different energy delivery parameters than an ablation made completely in an intercostal vein. To resolve this, the ablation catheter may have a plurality of ablation elements wherein at least one is fully positioned in an intercostal vein and the remainder may be in the intercostal vein or in the azygos vein or both. Different ablation energy delivery parameters may be used for the different scenarios, for example higher power or energy may be delivered to the ablation element in the azygos vein or ablation energy may only be delivered to the element(s) that are fully or partially in the intercostal vein. The location of the plurality of ablation elements may be determined with fluoroscopic imaging or by monitoring electrical impedance between each ablation element (e.g., RF electrode) and a dispersive electrode.

In some embodiments, two or even three levels may be ablated, which may further increase efficacy.

Alternative devices and methods of use may include a shorter ablation element that is used to create a relatively shorter ablation and repositioned a plurality of times to create multiple ablations within the GSN position range 68. In some embodiments, ablations may be made from the azygos vein, which may use different energy delivery parameters for example, higher energy or power.

An exemplary ablation catheter adapted to ablate a TSN (e.g., GSN) from an intercostal vein and or an azygos vein, for example using one or more of the embodiments of placement strategies disclosed herein, may have features that allow it to be delivered transvascularly to a desired location in a T9, T10, or T11 intercostal vein, be positioned relative to anatomical features to effectively ablate a target TSN while safely avoiding important non-target structures in a large majority of patients, and to deliver ablative energy capable of ablating the target TSN. The ablation catheter and system features may allow a user to ablate a TSN with relative ease and efficiency without sacrificing efficacy or safety. For example, once the ablation element(s) of the catheter are positioned (e.g., using methods disclosed herein), ablation energy may be delivered from a computerized ablation console with the press of a button or at least with minimal adjustments, repositioning, dragging, torqueing of the catheter or minimal user decisions regarding energy delivery. Features of ablation catheters and systems disclosed herein may allow a TSN/GSN to be ablated from one placement and energy delivery procedure or in some cases from an additional placement (e.g., in another of a T9, T10, or T11 intercostal vein) and energy delivery with a high probability of success in a large majority of patients.

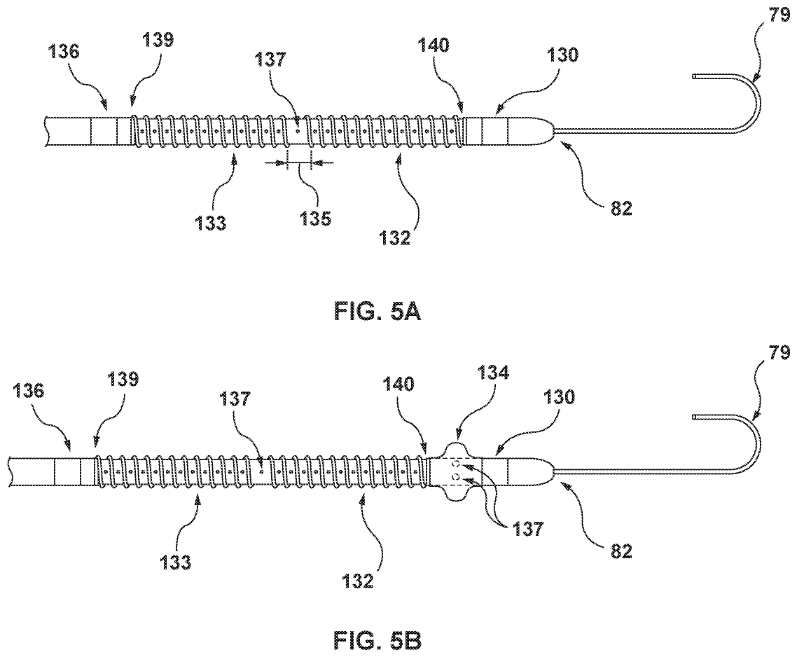

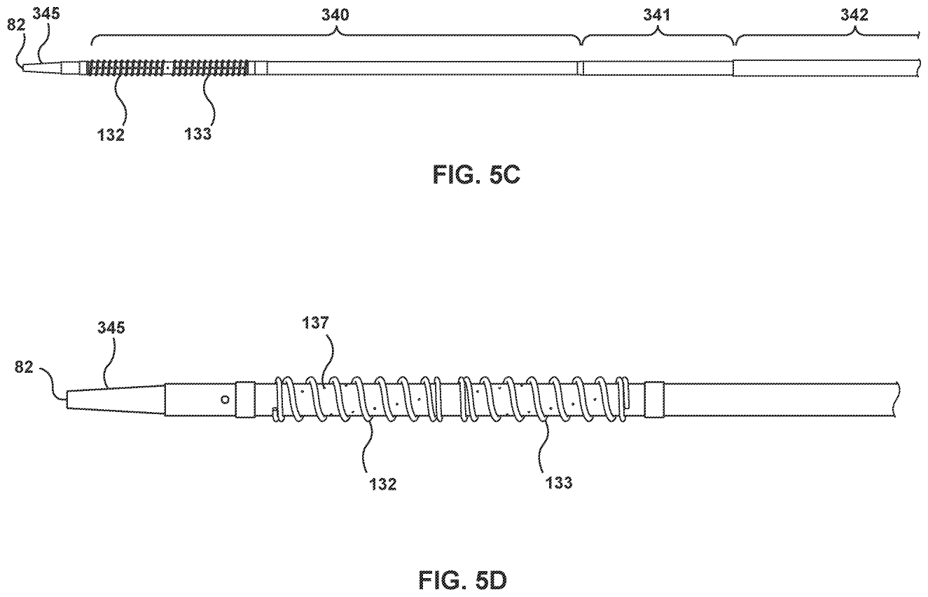

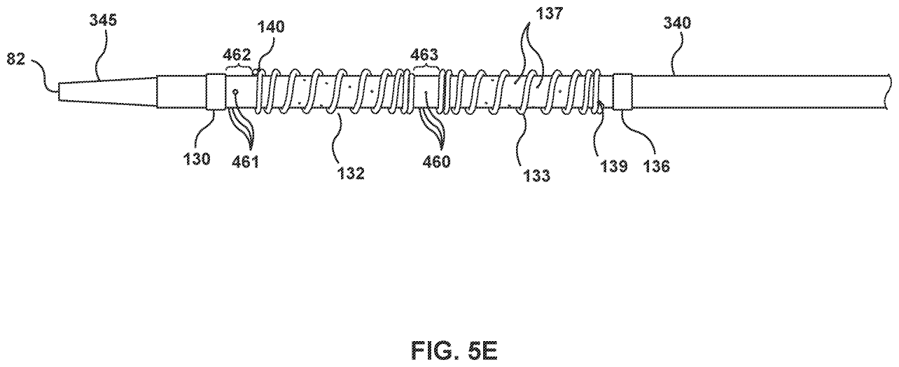

Exemplary ablation catheters that may be delivered to a target anatomical location for transvascular ablation (in some embodiments of a GSN) may have a proximal end, a distal end, an elongate shaft therebetween, a distal section (e.g., comprising the distal-most 7 cm), and an ablation element on, at or carried by the distal section. The ablation element may, in some embodiments, be adapted (including sized and/or configured) to create an ablation having a length in a range of 5 mm to 25 mm, preferably 10 to 25 mm (such as 15 mm to 20 mm) and a radial depth of at least 5 mm from the vessel surface. A handle may be located on the proximal end of the catheter to contain electrical or fluid connections or facilitate handling of the catheter. The elongate shaft from a strain relief region to the distal tip may have a length of 100 cm to 140 cm (such as from 110 cm to 130 cm, such as about 120 cm) allowing the distal section to be delivered from an arteriotomy such as a femoral vein access (or other access location such as jugular vein, brachial vein, radial vein, hepatic vein or subclavian vein) to a T11 intercostal vein in a large majority of human patients, or a length of 50 cm to 140 cm allowing the distal section to be delivered from a jugular vein access to a T11 intercostal vein in most patients. To be deliverable through a 9F delivery sheath (such as any of the delivery sheaths herein) the catheter may have a maximum outer diameter of 3 mm (e.g., 2.5 mm, 2 mm, 1.5 mm) at least in its delivery state. The catheter may in some embodiments have a deployable structure that expands beyond this dimension once advanced from the delivery sheath and positioned in a target vessel in some embodiments. An ablation catheter for delivering an ablation element to an intercostal vein, in particular a T9, T10 or T11 intercostal vein, from an endovascular approach including approaching the intercostal vein from an azygos vein may have a shaft with features that facilitate easy tracking over a guidewire, pushability, transfer of translation forces from the handle of the catheter, and passing over a tight bend from the azygos vein to the intercostal vein without kinking. As shown in FIG. 5C, the catheter shaft may comprise a first section 340, a second section 341 and a third section 342. The first section 340 may be more flexible than the second and third sections and may carry the ablation element such as two coiled electrodes 133 and 132 as shown. This first section may have a flexibility capable of passing over the tight bend from the azygos vein to intercostal vein (e.g., having a radius of curvature >=5 mm, and angle up to 120 degrees). The first section may have a length in a range of 60 mm to 100 mm (e.g., about 65 mm) and may be made from a single lumen Pebax® tube having a durometer from 50 to 60 D, such as 55D.

The second section 341 may have a flexibility between that of the first and third sections and function as a transition region and strain relief to resist kinking. For example, the second section may have a length in a range of 15 mm to 25 mm (e.g., about 20 mm) and may be made from a single lumen Pebax R: tube having a durometer from 60D-70D, such as from 60D-65D, such as 63D.

The third section 342 may be at least a portion of the proximal region of the elongate shaft and may be adapted for pushability, kink resistance, torque transmission, and flexibility. For example, the third section of the elongate shaft may span from the proximal end of the catheter to about 85 mm (e.g., in a range of 75 mm to 100 mm) from the distal end and may in some embodiments have a metal wire braid embedded into an outer layer of the shaft. An example material for the third section of the elongate shaft may be extruded Pebax R: having a durometer from 70D to 75D, such as 72D, for example. For example, the first section 340 may be more flexible than the second section 341 section, which may be more flexible than the third section 342 and flexibility may be increased by using a lower durometer material or more flexible braided outer layer or no braided outer layer. The maximum outer diameter of the elongate shaft, at least in a delivery state, may be in a range of 1.5 to 3 mm. In some embodiments, as shown in FIG. 5C, the first section 340 of the shaft may be made from a tube having a smaller diameter than the second section 341, which in turn may have a smaller diameter than the third section 342 of the shaft. For example, the first section may be made of a tube having an outer diameter of 2 mm; the second section may be made of a tube having an outer diameter of 2.5 mm; and the third section may be made of a tube having an outer diameter of 3 mm. In some embodiments, the elongate shaft may have a tapered, soft distal tip 345, which may have a length in a range of 5 mm to 30 mm (e.g., about 8 mm), and which may be softer than the first section. In some embodiments, the first, second, or third sections of the shaft may have a lubricious coating on the exterior surface to further improve delivery through vasculature. A guidewire lumen may pass through the elongate shaft with an exit port 82 at the distal tip of the shaft. The guidewire lumen may be made from, for example, a 0.014″ ID polyimide tube located in a lumen of the shaft.

Ablation catheters may, in some embodiments, have an ablation element adapted to deliver ablative energy to a target nerve up to 5 mm from the vessel surface for a total length in a range of 10 mm to 25 mm, such as 10 mm to 20 mm, such as 15 mm to 20 mm. The ablation element may be made of a plurality of ablation elements (e.g., two) positioned within a region of the shaft having a total length in a range of 10 mm to 25 mm, such as 10 to 20 mm, such as 15 mm to 20 mm even if the ablation elements are axially spaced apart. The ablation element(s) may include one or more of an RF ablation electrode, a coiled wire electrode, a laser cut RF electrode, an RF electrode printed with conductive ink, an RF electrode on an expandable balloon (e.g., made from conductive ink or flexible circuits), a conductive membrane RF electrode, an RF electrode on an expandable cage or mesh, an ultrasound ablation transducer, electroporation electrodes, a cryoablation element, or a virtual RF electrode.

The ablation element may be adapted to deliver ablation energy circumferentially, that is radially symmetric around the ablation element and around the vessel in which the ablation element is positioned. Although the GSN always passes anterior to the intercostal vein and azygos, it is safe and acceptable to ablate tissue around the intercostal or azygos veins, and ablating circumferentially may allow for a simpler and faster procedure that is also less prone to user error because aiming the energy delivery is not necessary. Features that may allow for circumferential ablation may include, without limitation, ablation electrodes that expand to contact the vessel wall evenly around the circumference of the vessel, ablation electrodes that are used with an electrically conductive fluid, electrically insulative balloons or deployable structures that contain ablative energy in a segment of a target vessel allowing it to be directed radially, ablation elements that direct ablation energy circumferentially such as cylindrical ultrasound transducers.

In some embodiments, the ablation element is an RF electrode and saline may be delivered to the vessel in fluid communication with the RF electrode. An irrigation lumen in communication with irrigation ports may located distal to the ablation element, under the ablation element (in some designs where irrigated saline can pass through the ablation element), or in a deployable structure in some embodiments). An irrigation lumen may be for example a lumen in the elongate shaft in fluid communication with a tube on the catheter's proximal end that is connectable to a fluid source and pump.

In some embodiments, at least one deployable occlusive structure (e.g., balloon, bellows, wire mesh, wire braid, coated wire mesh, or coated wire braid) may be positioned on the shaft distal to the ablation element. The deployable structure may function to anchor the catheter in place during energy delivery and possibly to improve safety by avoiding ablation of the sympathetic trunk by providing an electrical insulator or containing saline proximal to the deployable structure. In some embodiments, a deployable occlusive structure may be located just proximal to the proximal end of the ablation element(s) which may function to divert blood flowing in the azygos vein away from the ablation zone. For example, a deployable occlusive structure may be a balloon such as a urethane balloon having a length (along the axis of the shaft) of about 2.5 mm and an inflated diameter of about 2.5 mm to 7 mm (e.g., 3 mm to 6 mm, 4 mm to 5 mm). The balloon may be in fluid communication with an inflation port connecting the balloon with an inflation lumen connectable to an inflation source on the proximal end of the catheter. In some embodiments, the inflation lumen may be in fluid communication with an irrigation lumen connectable to an irrigation source and pump. In some embodiments such a catheter may have a balloon with holes that allow irrigation fluid to exit the inflated balloon and flow toward the ablation element(s).

Ablation catheters may, in some embodiments, have a proximal radiopaque marker positioned on the shaft at or proximal to the proximal end of the ablation element(s). In some embodiments, ablation catheters may include a distal radiopaque marker which may be positioned on the shaft at or distal to the distal end of the ablation element. In some embodiments, there may be a space between a distal radiopaque marker and the distal end of the ablation element, the space having a length in a range of, 1 mm to 25 mm, such as, 1 mm to 5 mm, such as, 1 mm to 3 mm, such as 0.5 mm, 1 mm, or 1.5 mm. For example, as shown in FIG. 2 a distal radiopaque marker 130 may be aligned with or positioned relative to an anatomical landmark such as the costovertebral joint 61 and a space 135 (e.g., 0.1 mm to 25 mm) is between the distal radiopaque marker 130 and the distal end of the ablation element 132 ensuring the ablation element is safely distant from the sympathetic trunk 54. In some embodiments, a deployable structure 134 may be positioned in the space transitionable between a contracted state (OD similar to the shaft OD e.g., in a range of 1.5 mm to 3 mm) and deployed state (OD increases to a range of 3 to 7 mm). The deployable structure may be a balloon, bellows, wire mesh, wire braid, coated wire mesh, or coated wire braid.

An example of an ablation catheter that is sized and adapted for GSN ablation is shown in FIG. 2. Ablation catheter 81 has an elongated shaft sized and adapted to reach a T1l intercostal vein from an introduction site at a femoral vein or jugular vein. The distal section of catheter 81, shown positioned in an intercostal vein 55, includes a distal radiopaque marker 130 that is aligned with or positioned relative to a costovertebral joint 61, an ablation element 131 comprising or consisting of a distal conductive coiled RF electrode 132 and a proximal conductive coiled RF electrode 133, an optional inflatable balloon 134 disposed between the ablation element 131 and the distal radiopaque electrode 130. The distal radiopaque marker 130 is in some embodiments spaced distally apart from the distal end of the ablation element 132 by a distance 135 for example in a range of 0 to 25 mm (e.g., such as a range of, 1 mm to 20 mm, such as a range of, 1 mm to 15 mm, a range of, 1 mm to 3 mm, such as 0.5 mm, 1 mm, or 1.5 mm). Catheter 81 also includes a proximal radiopaque marker 136 that is located at or near a proximal edge of the ablation element 131. In some embodiments proximal radiopaque marker 136 is axially spaced between 0) mm and 25 mm from a proximal end of ablation element 31 (which may be from a proximal end of ablation element 133).

The exemplary axial distances between markers and electrodes described herein (e.g., 0 mm to 25 mm, or 0 mm to 15 mm) may be integrated into any other ablation catheter herein unless indicated herein to the contrary.

Ablation electrodes 132 and 133 (or any other ablation electrode herein) may be made from, for example. Nitinol wire coiled around the catheter shaft, which may allow the electrodes to be flexible so they can traverse a tight bend from the azygos vein to the intercostal vein and also create a long ablation (e.g., 5 to 25 mm). Nitinol is an example of a superelastic material that allows the ablation element(s) to bend when traversing anatomical bends, and then elastically return to a linear or straight configuration once the electrode is past the bend.

Any of the distal sections herein may thus be described as a distal section that has an at-rest (as manufactured) linear or straight configuration. This would be in contrast to distal sections that may revert or assume non-linear at-rest configurations (e.g., a distal section with electrodes thereon that returns to a coiled configuration).

In some embodiments, the ablation catheter 81 includes at least one irrigation port 137 (as shown in FIG. 2) in fluid communication with an irrigation lumen that is near the coil electrodes for delivering a fluid such as saline. Saline delivery may facilitate delivery or removal of the device, or can be used during energy delivery to improve ablation formation and prevent overheating, for example. In some embodiments, catheter 81 may include a guidewire lumen 82 for delivery over a guidewire 79.

FIG. 5A illustrates a portion of an exemplary ablation catheter, including at least a portion of a distal section thereof. The ablation catheter in FIG. 5A includes an ablation element that includes a distal ablation element and a proximal ablation element. The ablation element (and other ablation elements herein) includes or consists of a distal conductive coiled RF electrode 132 and a proximal conductive coiled RF electrode 133, as shown in FIG. 5A. Both distal and proximal coiled electrodes may be helical coils positioned around and at least partially on the outer surface of the shaft, in some embodiments in a groove in the shaft. The coiled electrodes may be helical, and may have varying directions, pitches, or wire thickness, and may be made from a round wire or ribbon wire of electrically conductive material such as stainless steel or superelastic Nitinol, in some embodiments electropolished, in some embodiments including a radiopaque material such as platinum iridium. Alternatively, one or more coiled electrodes may be made from a laser cut tube such as a Nitinol tube forming a coiled pattern or other flexible pattern. Alternatively, the ablation element (e.g., ablation element 131) may be made from a distal and a proximal flexible electrode in the form of wire mesh or braid. Alternatively, the flexible ablation element may comprise a plurality of ring electrodes each having a length no more than 5 mm, such as 3 mm. In some embodiments, the flexible ablation element may have an expandable diameter transitionable from a contracted delivery state to an expanded deployed state (e.g., having an outer diameter up to about 5 mm) so it can expand to contact the vessel wall.

Electrodes herein, such as the proximal and distal electrodes herein (e.g., distal electrode 132 and proximal electrode 133) may have a length that is in a range of 4 mm to 12 mm, such as 5 mm to 11 mm, and in some embodiments they are or about 5 mm, 5.5. mm, 6 mm, 6.5 mm, 7.0 mm, 7.5 mm, 8 mm, 8.5 mm, 9 mm, 9.5. mm, 10 mm, 10.5 mm, or 11 mm. Proximal and distal electrodes may have the same or substantially the same lengths, including lengths that are in the ranges provided herein (e.g., 5 mm to 11 mm). In some embodiments electrodes may have different lengths. For example, in some examples distal electrode 132 may be longer than proximal electrode 133, but the electrodes individually may have any of the lengths herein. In some examples distal electrode 132 may be shorter than proximal electrode 133, but the electrodes individually may have any of the lengths herein.

For catheters that have a plurality of electrodes, each electrode may be connected to an independent conductor passing through the elongate shaft to the proximal region of the catheter where it is connectable to an extension cable or ablation energy source. This can allow each electrode to be independently energized in monopolar mode or bipolar mode.

For some catheters with distal and proximal electrodes, the catheters may include a gap between a distal end of the proximal electrode and a proximal end of the distal electrode. In some embodiments the gap may be in a range of 0) to 5 mm, such as 0 mm 4 mm, such as, 1 mm to 1.25 mm, such as 0.25 mm, 0.5 mm, 0.75 mm, 1 mm, or 1.25 mm. Preferably the proximal and distal electrodes are not in electrical communication with one another. Alternatively, the proximal and distal electrodes may at least partially overlap one another along their lengths, as long as they are not in electrical communication with one another.

A gap between proximal and distal electrodes may be such that it is not so large that it prevents a continuous ablation lesion to be formed. Gaps described herein (e.g., 0 mm to 5 mm, such as, 1 mm to 1.25 mm, such as 0.25 mm, 0.5 mm, 0.75 mm, 1 mm, or 1.25 mm) can provide the exemplary benefit of providing for continuous lesion formation.

Ablation catheters herein may include one or more temperature sensors. FIG. 5A illustrates an exemplary ablation catheter that comprises at least one temperature sensor. The ablation catheter shown includes, for example, a proximal temperature sensor 139 that may be positioned in contact with proximal electrode 133, and in some embodiments on the proximal end of proximal electrode 133. The ablation catheter shown also includes a distal temperature sensor 140 that may be positioned in contact with distal electrode 132, and in some embodiments on the distal end of the distal electrode. Any of the ablation catheters herein may in some embodiments include another temperature sensor that may be positioned between proximal and distal electrodes, or between a plurality of electrodes. For catheters that include one or more temperature sensors, the temperature sensor(s) may be thermocouples (e.g., T-type) or thermistors. In some embodiments, at least one temperature sensor may radially extend or be radially extendable from the catheter shaft to contact tissue up to 3 mm away from the catheter surface. The temperature sensor(s) may be connectable at the proximal region of the catheter to a computerized energy delivery console where signals from the sensors may be input and used in an energy delivery control algorithm.

Any of the ablation catheters herein may include one or more irrigation ports (which may be referred to herein as holes or apertures) in fluid communication with an irrigation lumen that is connectable to a fluid source at the proximal region of the catheter for delivering a fluid such as saline (e.g., normal or hypertonic saline) to the vessel. The ports may be formed in one or more layers of the elongate shaft to create the fluid communication between the port and the irrigation lumen. The fluid may function to cool or remove heat from the electrode(s) and/or vessel wall, to flush blood from the vessel to reduce risk of clot formation or improve ablation consistency, to conduct electrical energy from the ablation electrodes, to control pressure in the vessel, to facilitate delivery of the distal section of the ablation catheter to a target vessel (e.g., intercostal vein), or to facilitate removal of the distal section of the ablation catheter from the target vessel. In some embodiments, one or more irrigation ports may be distal to the ablation element(s), or distal to each of the plurality of flexible ablation elements. In some embodiments, any of the irrigation port(s) may be positioned radially under the flexible ablation element(s). In some embodiments, one or all irrigation ports may be disposed between windings of coiled ablation element, such that the port is not radially under the winding of the ablation element. In some embodiments, an irrigation port may be positioned in an axial gap or space between adjacent ablation electrodes. In some embodiments, one or more irrigation ports may be in a cavity of a deployable occlusive structure (e.g., balloon) and may function to inflate the balloon, wherein the balloon may have a perforation on its proximal side that allows the fluid to escape the balloon into the target region of the vessel.

FIGS. 5A-5E illustrate a distal section of a merely exemplary ablation catheters, which in this embodiment includes a plurality of irrigation ports between windings of coiled ablation elements (although only one port 137 is labeled, the others can be seen in the figures).

In some embodiments, as shown in FIG. 5D, irrigation holes (which may be referred to herein as apertures or ports) 137 may be positioned between windings of the coil electrodes and be circumferentially distributed to deposit saline along the length of the ablation electrodes as well as circumferentially around the electrodes.

FIG. 5E is a schematic illustration of a distal portion of an ablation catheter, wherein irrigation holes 137 may be arranged in a helical pattern between at least some windings of a proximal helical electrode 133 and likewise irrigation holes 137 may be arranged in a helical pattern between at least some windings of a distal helical electrode 132, and a plurality of irrigation holes 461 may be arranged distal to the distal electrode and a plurality of irrigation holes 460) between the proximal and distal electrodes.

In some embodiments, the ablation catheter may have a deployable element transitionable from a contracted delivery state (e.g., having an OD in a range of 1.5 mm to 3 mm) to an expanded deployed state (e.g., having an OD in a range of 2.5 mm to 6 mm) that functions to one or more of anchor the distal section of the catheter in the target region of the vessel, to occlude blood flow, to contain delivered fluid such as saline, to maintain vessel patency, or to act as an electrical insulator. For example, as shown in FIG. 5B, any catheter herein may also include a distal deployable element 134 coupled with optimized irrigation flow that may create a virtual electrode that provides an effective ablation without the need for wall contact. Distal deployable element 134 may be a balloon (e.g., compliant balloon) as shown in FIG. 5B, or alternatively a bellows or coated stent or mesh. Distal deployable element 134 is distal to the ablation element, which may include proximal and distal electrodes as shown in FIG. 5B.

The disclosure above described exemplary methods of positioning an ablation catheter within an intercostal vein to ablate a GSN while minimizing or avoiding damage to non-target structures. The ablation catheter shown in FIGS. 5A-5E included one or more radiopaque markers (e.g., distal marker 130 and proximal marker 136) that can be used as part of those methods of positioning. While the ablation catheter in FIGS. 5A-5E is an example of an ablation catheter that may be used when performing methods herein, it is understood that the methods may be performed with a variety of ablation catheters. It is thus understood that the methods herein are not limited by the particular ablation catheters herein. It is also understood that the ablation catheters herein need not be used with the positioning methods herein.

Alternative embodiments of TSN/GSN ablation catheters may have one or more the features that are described herein, such as proximal and distal radiopaque markers spaced as described, irrigation lumens(s), temperature sensor(s), guide wire lumens, flexible shaft section, and may also include alternative ablation elements. For example, ablation elements may be RF electrodes having different configurations or ablation elements that deliver a different type of ablation energy such as ultrasound, electroporation, cryoablation, laser, chemical or other ablation modality. Ablation catheter features that are described with respect to one embodiment or example herein may be incorporated into other suitable embodiments unless the disclosure indicates otherwise. Features with the same or similar reference numbers are understood to be in some embodiments included and can be the same component.

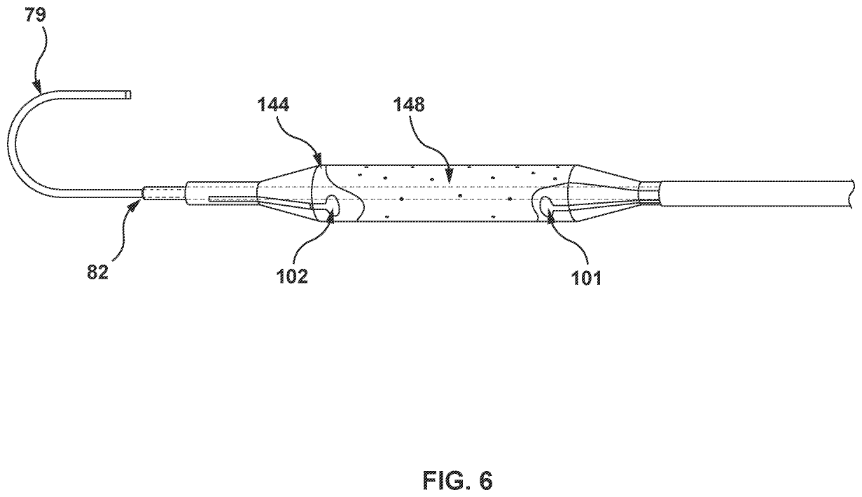

FIG. 6 illustrates an exemplary ablation catheter with ablation element(s) carried by an expandable balloon. FIG. 6 illustrates a distal section of an ablation catheter with an RF ablation element, wherein the ablation element includes one or more electrically conductive element(s) positioned on expandable balloon 144. The conductive elements may be a film or conductive ink or flexible circuits. Sensors (e.g., temperature sensors) may be positioned on the balloon as well. In some embodiments the balloon may be inflated by delivering fluid such as saline or air into the balloon. In some embodiments, the conductive element(s) or the balloon may have perforations allowing fluid to pass through to cool the electrode or conduct energy. The pattern of the conductive element(s) may be cylindrical 148.

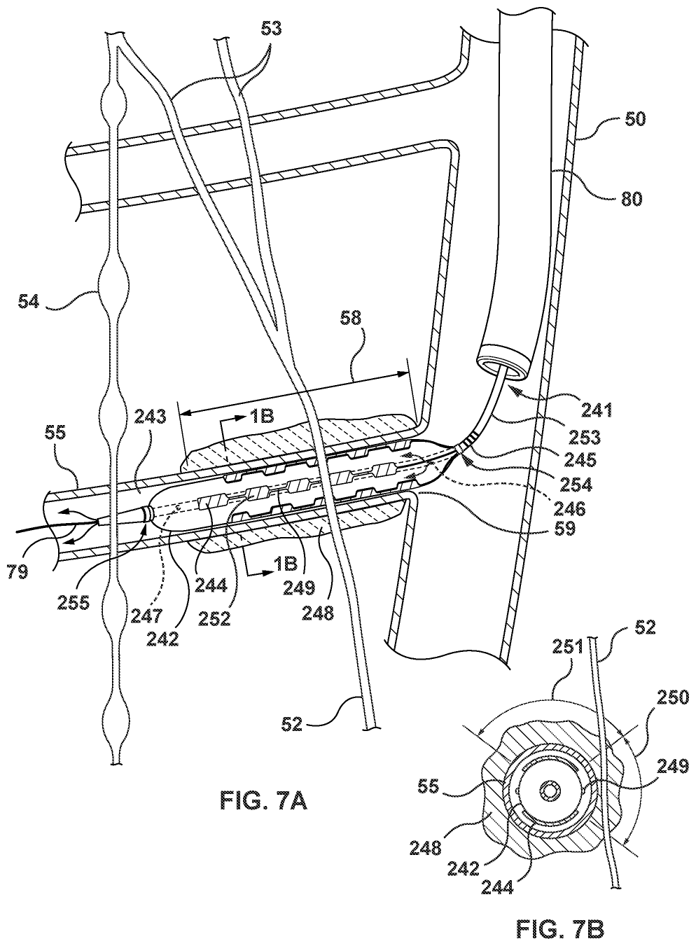

Another embodiment of a transvascular ablation catheter 241 for ablating a TSN or GSN from within an intercostal nerve is shown in FIG. 7A. The catheter 241 may extend along a longitudinal axis. An expandable member, for example in the form of a balloon 242 having an unexpanded state and an expanded state, may be coupled to a distal section 243 of the catheter. The expandable member (e.g., balloon) may have a circumferential treatment zone 248 (e.g., having a length in a range of 5 to 25 mm, in a range of 10 to 15 mm) extending along the longitudinal axis in the expanded state and surrounding the vessel 55. The catheter includes an electrode assembly 252, which comprises a plurality of electrode pads 244, may be mounted or otherwise secured to the balloon 242. Each electrode pad assembly may include a substrate supporting first and second electrode pads with each electrode pad having a pair of elongate bipolar electrodes and connected with an electrical trace 249. The electrode pads of each electrode pad assembly may be longitudinally and circumferentially offset from one another. The method may also include expanding the balloon in the intercostal vein so as to electrically couple the electrodes with a wall of the intercostal vein and driving bipolar energy between the electrodes of each bipolar pair so as to therapeutically alter the TSN or GSN within 5 mm of the intercostal vein such that the blood volume of the patient is redistributed for treatment of diseases such as pulmonary hypertension, or heart failure (e.g., HFpEF).

Each electrode pad may include a temperature sensor disposed between the electrodes of the pair. The expanding of the balloon may couple the temperature sensors with the wall of the intercostal vein. In some embodiments, the method may further include directing the energy to the bipolar pairs in response to a temperature signal from the temperature sensor so as to heat the wall approximately evenly.

To create an ablation having a depth of 5 mm to target a GSN from an intercostal vein the electrode pads may be cooled to allow greater power to be delivered without desiccating tissue of the vein wall, which impedes ablation depth. The electrodes may be cooled for example, by circulating coolant in the balloon 242. In one embodiment coolant may be injected into the balloon 242 from a coolant injection port 246 at one end of the balloon chamber and the coolant may exit the chamber through an exit port 247 at the opposing end of the chamber and allowed to return through the catheter through an exit lumen.

Electrode pads may be positioned around the balloon to make a circumferential ablation pattern that is as long as the target ablation zone 58 (e.g., up to 20 mm, about 15 mm, between 12 and 18 mm). For example, as shown in FIG. 67B, a balloon with electrode pads mounted to an elongate shaft 253 may have an undeployed state having a diameter of about 1 mm to 2.5 mm and a circumference of about 3.14 mm to 7.85 mm and be expandable to a deployed state having a diameter in a range of about 3 mm to 5 mm and a circumference in a range of about 9.4 mm to 15.7 mm. Electrode pads 244 may be separated or spaced by a distance 250 of less than 5 mm (e.g., less than 2.5 mm) and width or arc length 251 in a range of 3 mm to 3.5 mm. Electrode pads 244 may have a length of about 3 to 5 mm each. As shown in FIG. 67A, an electrode pad assembly 252 may comprise multiple electrode pads 244 arranged on four separate rows connected together by electrical traces 249, the rows evenly spaced around the circumference of the balloon 242 (e.g., four rows at each 90-degree quadrant). Longitudinally, the pads 244 on one row may be offset from pads of adjacent rows. When the balloon is in its unexpanded state the space between the electrode pads is decreased (e.g., to about 0 to 1 mm) and the adjacent rows interlock with one another. In its expanded state the space 250 between the pads expands due to the expandable balloon 242 to about 2 mm to 5 mm. The balloon 242 may be a compliant material such as latex or a non-compliant material that flexibly folds to contract.

Just proximal to the balloon the catheter shaft may comprise a flexible neck 245 that allows the ablation balloon to sit in the intercostal vein's natural orientation. Given the small bend radius at this location a stiff shaft could apply force to the ablation balloon causing it to distort the intercostal vein and reduce predictability of ablation zone. A flexible neck may be made of a softer durometer polymer (e.g., Pebax R) and may have a wire coil embedded in the material, which may allow flexible bending while providing pushability. This type of flexible neck may be incorporated into other ablation catheters herein.

The electrode(s) that are most proximal may be placed just in the intercostal vein near the ostium. Blood flow through the azygos vein may metabolically cool tissue near it impeding ablation creation. A larger amount of ablation power (e.g., RF) or longer duration may be delivered to this proximal electrode(s) than the rest of the electrode(s) to compensate for the blood flow cooling.

The catheter 241 may have a distal radiopaque marker 255 positioned distal to the ablation elements, for example distal to the balloon 242, and/or a proximal radiopaque marker 254 positioned proximal to the ablation elements 244, for example proximal to the balloon 242. The distal and proximal radiopaque markers 255, 254 may be separated along the longitudinal axis of the shaft by a distance in a range of 5 mm to 25 mm (e.g., 10 mm to 15 mm). Any other features or description of radiopaque markers herein may apply to markers 255 and/or 254.

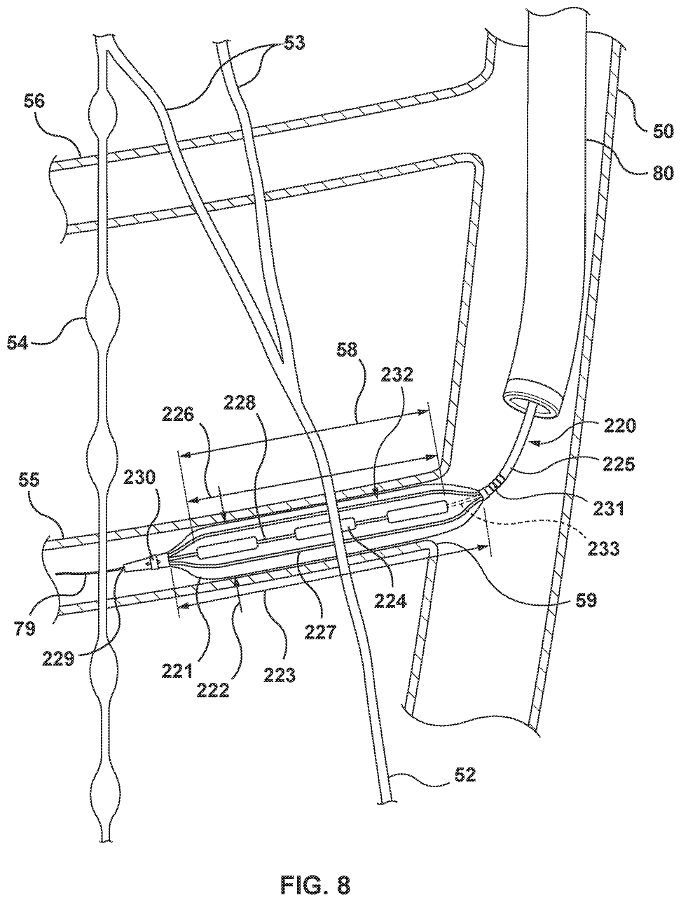

FIG. 8 illustrates an exemplary ultrasound ablation catheter. Catheter 220 includes an elongate shaft 225 with a proximal region and a distal section and an ablation assembly 232 mounted to or at the distal section. The ultrasound ablation catheter 220 has an inflatable balloon 221 which may have a geometry suitable for expansion in an intercostal vein (e.g., outer diameter 222 in a range of 2.5 to 5 mm in its inflated state) and a length 223 in a range of 8 to 30 mm. Within the balloon 221, multiple ultrasound transducers 224 are positioned on a shaft 233 centered in the balloon 221. The transducers 224 may be placed serially spanning a length 226 that is in a range of 5 to 25 mm to generate an ablation of a similar length capable of creating an ablation the length of the target ablation zone 58. Due to the small diameter of the intercostal vein the reduced balloon size may risk contacting the transducer or getting over heated by the transducer, which may rupture the balloon or reduce efficacy of the ablation. To remedy this risk struts or protrusions 227 may be positioned between the transducer and balloon. The struts 227 may be for example polymer strands elastically pre-shaped to radially expand away from the transducers 224. To make a longer ablation to span the targeted ablation zone, multiple transducers may be incorporated (e.g., three 4 mm long transducers) and spaced apart with flexible gaps 228 between them to facilitate traversing the small bend radius from the azygos vein to intercostal vein. For example, shaft 225 may be a braid reinforced polyimide tube with an optional guidewire lumen 229 for delivery over a guidewire 79 and carry electrical conductors that energize the transducers 224. The ultrasound transducers 224 may be cylindrical for producing circumferential ablation around the target vein. Alternatively, the ultrasound transducers may be flat or hemicylindrical to produce an ablation that is a partial segment of the circumference of the vein and a radially identifiable radiopaque marker 230 may be positioned on the distal section allowing a user to orient the direction of ablation toward the patient's anterior where the GSN passes over the vein 55. In some embodiments, the ultrasound transducer may be configured to image as well as ablate and the imaging function may be used to assess nearby structures such as the lung, vertebra, ribs. Imaging ultrasound may be used to confirm the transducer is aiming toward the lung, which is the direction of the target GSN. In some embodiments, the shaft may have a flexible neck 231 within 10 mm proximal of the balloon 221 to allow the distal section to sit well in the intercostal vein.

In an alternative embodiment of an ultrasound ablation catheter, the catheter can be composed of an active ultrasound transducer and an inflatable reflector balloon, which may be on the same catheter or alternatively be on separate catheters. The reflector balloon may have an inflated diameter in a range of 2.5 to 4 mm and on its proximal surface have a shape such as a concave curvature that focuses reflected waves on to the target ablation zone. The reflector balloon is located distal to the transducer and is inserted in the narrower intercostal vein, while the ultrasound transducer remains in the larger azygos vein. The ultrasound transducer may be exposed to blood flow in the azygos vein or alternatively may be contained in a chamber in an inflatable balloon filled with coolant (e.g., circulating coolant such as sterile water or saline). The ultrasound energy is directed toward the distal reflector balloon and reflected and focused into tissue surrounding the splanchnic nerve. The advantage of this approach is that an active ultrasound transducer can be made larger and is not required to go through the sharp turn from azygos to intercostal vein. A second advantage is that several intercostal veins can be used to target ablation with the same catheter.

The catheter 220 may have a distal radiopaque marker 230 positioned distal to the ablation elements, for example distal to the balloon 221 and a proximal radiopaque marker positioned proximal to the ablation elements, for example proximal to the balloon. The distal and proximal radiopaque markers may be separated along the longitudinal axis of the shaft by a distance in a range of 5 mm to 25 mm (e.g., 10 mm to 15 mm).

In some methods of use, the ablation energy is RF, and an energy delivery controller is adapted to deliver RF power in a range of 15 W to 50 W. In some embodiments, the controller is adapted to deliver RF power in a range of 15 W to 40 W, in a range of 15 W to 35 W, or in a range of 20 W to 35 W, such as about 25 W, about 30 W or about 35 W.

Some of the devices herein may have one or more features that provides for a safe delivery to the target vessel.

Some of the devices and methods of use herein may safely deliver energy with temperature monitored energy delivery.

Some of the methods of use herein may generate a lesion capable of targeting a nerve up to 5 mm away from the target vessel and within a target region having a continuous lesion length from 5 mm to 25 mm, such as 10 mm to 25 mm, such as 15 mm to 20 mm. (e.g., 15 mm, 16 mm, 17 mm, 18 mm, 19 mm, 20 mm), with a single positioning and delivery of energy.

Some of the devices and methods herein are adapted to avoid risks of boiling, hot spots, or erratic energy delivery that could decrease ablation efficacy. Furthermore, some embodiments may include nerve stimulation to identify a target nerve or non-target nerve to confirm positioning prior to ablation, or to confirm technical success during or following ablation.

It may be preferred, but not required, that the methods of ablation create a continuous ablation zone (i.e., not having separate, discrete regions of ablated tissue that are not connected to each other). This ensures that the region of tissue where the target GSN nerve or GSN nerve root is likely to be located is most likely to be effectively ablated by the ablation energy. The continuous ablation zone may be circumferential, or less than circumferential.

In some embodiments, an ablation confirmation test can then be performed, for example, by delivering a nerve stimulation signal. Monitoring can be performed for a physiological response (e.g., splanchnic vasoconstriction, increased heart rate, increased blood pressure) to the ablation confirmation test. If the physiological response demonstrates that the first lesion did not provide a clinically significant amount of GSN blocking (e.g., by observing a lack of physiological response) then ablation energy can be delivered from the ablation catheter to create a second lesion in tissue up to 5 mm from the second intercostal vein. The distal section of the ablation catheter can be moved to a third intercostal vein that is superior to (e.g., superior and adjacent to) the second intercostal vein. The same or different ablation confirmation test can be performed, followed by another monitoring test. If the physiological response demonstrates that the first lesion and second lesion did not provide a clinically significant amount of GSN blocking (e.g., by observing a lack of physiological response) then ablation energy can be delivered from the ablation catheter to create a third lesion in tissue up to 5 mm from the third intercostal vein. Any of the the ablation confirmation tests may comprise delivering a nerve stimulation signal from a stimulation electrode positioned on the distal section of the ablation catheter configured to generate an action potential in the thoracic splanchnic nerve. Alternatively or in addition to, the ablation confirmation test may comprise a leg raise test. Alternatively or in addition to, the ablation confirmation test may comprise adding fluid volume to the venous system. Alternatively or in addition to, the ablation confirmation test may comprise a hand-grip test. Alternatively or in addition to, the ablation confirmation test may comprise measuring venous compliance or capacitance.

In exemplary methods in which an ablation confirmation test includes a leg raise test, the method may comprise any of the following steps. Prior to ablation in the lowest intercostal vein, a baseline measurement may be obtained by raising the legs and measuring the change in central venous pressure and waiting for equilibration, that is a measure of the total venous compliance including the central veins and splanchnic bed. The legs can then be lowered, to allow equilibration so blood redistributes back to the legs. An ablation in the lowest intercostal vein (e.g., T11) can then be performed as set forth herein. The legs can then be raised, followed by waiting for equilibration and re-measure central venous pressure. A measurement can then be made to determine if there was an appropriate reduction in total venous compliance. If yes, then the GSN has successfully been ablated. If no, then an ablation in the next higher intercostal vein (e.g., T10) can be performed, as set forth herein. The measurement can be repeated. A determination can then be made to see if there was an appropriate reduction in total venous compliance. If yes, then the GSN has successfully been ablated. If no, then an ablation in the next higher intercostal vein (e.g., T9) can be performed.

In exemplary methods in which an ablation confirmation test comprises a hand-grip or other activity that increases sympathetic nervous system (SNS) outflow to the splanchnic bed may comprise the following steps. An ablation can be performed in a lowest intercostal vein (e.g., T11). Venous compliance can then be measured. A hand-grip can then be performed for a predetermined amount of time (e.g., 60) seconds). Venous compliance can then be remeasured. If there is no change in venous compliance, the initial ablation was sufficient to achieve a clinically significant outcome. If there still is a decrease in compliance, some of the SNS activity caused by the hand-grip is getting through. The ablation in the lowest intercostal vein was thus insufficient to achieve a clinically significant effect. An ablation in the next higher intercostal vein (e.g., T10) can then be performed. A hand grip test for a predetermined amount of time (e.g., 60 seconds) can be performed. Venous compliance can then be remeasured. If there is no change in compliance, the second ablation was sufficient. If there is a decrease in compliance, some of the SNS activity caused by the hand-grip is getting through, and the ablation in the next higher intercostal vein was thus insufficient to achieve a clinically significant effect. Ablation is the next higher intercostal vein (T9) can then be performed. The procedure is done at this point as ablation at a level higher than the 3rd lowest intercostal vein is not anticipated.

### Energy Delivery Algorithms

One aspect of the disclosure herein is related to energy delivery algorithms that are adapted to be particularly suited for ablating tissue circumferentially around a narrow blood vessel such as an intercostal vein or other similar vessel to a depth of at least 5 mm and up to 10 mm and from an ablation catheter. The ablation catheter may be any of the catheters herein or any other suitably adapted catheter. The methods of energy delivery below are understood to be merely illustrative and are non-limiting.

A first embodiment of an exemplary energy delivery algorithm is referred to as “Multiplexed Monopolar RF”, wherein pulses of RF are delivered to the plurality (e.g., two) electrodes in monopolar configuration with asynchronous waveforms. Each electrode receives a pulsed waveform of RF energy alternating on and off at a steady frequency. The waveforms may be for example square wave, sinusoidal, or other form of alternating waveform. The on period delivers an ablative level of RF power while the off period delivers a non-ablative level of RF power (e.g., in a range of 0 W to 1 W, about 0.1 W). The waveforms for each electrode are asynchronous, that is to say the waveforms are aligned in time so that an on period for one electrode is aligned with off periods of the remaining electrode(s) and vice versa.

An alternative embodiment of an Ablation Energy Delivery Algorithm used to create a desired lesion for GSN ablation, is referred to as “Sequential Monopolar with Bipolar Fill”, wherein ablative RF energy is delivered in monopolar mode to a first ablation electrode (e.g., the distal electrode 132 shown in FIGS. 1, 2, 5A-5E) for a First Electrode Monopolor Duration, then to a second ablation electrode (e.g., the proximal electrode 133) for a Second Electrode Monopolar Duration, then ablative RF energy is delivered in bipolar mode between the first and second electrodes for a Bipolar Duration and with an Initial Bipolar Power. If temperature measured by a temperature sensor associated with the electrode receiving ablation energy raises above an Upper Monopolar Temperature Limit the Initial Monopolar Power of RF energy may be decreased to a Secondary Monopolar Power or alternatively be decreased by a Power Decrement. If the temperature rises above the upper Temperature Limit again while the lower power is being delivered then the power may be decreased again, either to a Tertiary Power or by the Power Decrement. In some embodiments, a user may define parameters such as Initial Power to each ablation electrode. First and Second Electrode Monopolor Durations. Power Decrement or Secondary. Tertiary etc Monopolar Power. Likewise, during the Bipolar phase the Initial Bipolar Power may be decreased to a Secondary Bipolar Power or by a Power Decrement if measured temperature from either of the temperature sensors associated with the activated electrodes rises above an Upper Bipolar Temperature Limit.

The disclosure that follows provides some exemplary methods of use and steps thereof. Some embodiments of a method of use may include one or more of the following steps, the order of which may in some instances be varied, and not all steps of which need necessarily be performed. Methods herein may include interventional access, which may include one or more of the following treat the patient with an anti-coagulation regimen that is appropriate for venous interventional procedures; place a return electrode on the patient's right chest; follow standard techniques for femoral, subclavian, or jugular vein puncture, guide wire insertion, and sheath placement using heparinized saline where appropriate; place 0.035 exchange length guide wire (e.g., Cordis Amplatz. Super Stiff 260 cm or equivalent); advance a 6F general purpose catheter (e.g. JR4 or equivalent) over the guide wire to the azygous vein ostium; using the 6F general purpose catheter, inject a bolus of radiopaque contrast to identify the azygos vein ostium using fluoroscopy; engage the azygos vein ostium with the guide wire and 6F general purpose catheter and advance the guide wire through the valve (if applicable) into the azygos vein; exchange the 6F general purpose catheter for an azygos access sheath, wherein the azygos access sheath may be 9F and at least 100 cm long (e.g., Arrow 9F Super Arrow Flex Introducer Sheath or equivalent): position the azygos access sheath approximately to the T9 level: adjust the C-arm off the vertical axis to obtain the optimal view of the azygos vein tree via shooting contrast prior to introduction of the Ablation Catheter; load a 0.014 exchange length guide wire (e.g. ChoICE Pt LS Floppy or equivalent) into the azygos access sheath; and advance the 0.014 guide wire and deep seat into a first target intercostal vein (e.g., T1l intercostal vein).

Methods herein may include device, generator, and accessory preparation, which may include one or more of the following steps: inspect the catheter package prior to use; open the Ablation Catheter package using sterile technique; while maintaining sterility, remove the Catheter from its package and place in a sterile field; visually inspect the electrodes and ablation catheter carefully for integrity and overall condition; fill a 10 cc or larger syringe with saline and connect the syringe to the guidewire lumen hub on the handle of the ablation catheter. Flush the guidewire lumen with the saline to remove all air; prepare the ablation catheter by connecting the ablation catheter irrigation line to a 3-way stopcock, connecting the tube set to the 3-way stopcock and connecting the saline spike on a hanging sterile saline bag, and ensuring the stopcocks on the saline inlet and saline outlet lines are in the open position; place the irrigation pump tubing into the pump, through the bubble detectors and close the pump door; power ON the Generator (also referred to as a computerize console) and initialize the pump; flush the irrigation lumen of the ablation catheter using the pump to pump the saline through the irrigation lumen; confirm that the irrigation ports are patent; purge the tubing and ablation catheter of air bubbles; watch the saline tubing and Catheter tip for bubbles and continue to de-bubble until there is no air in the ablation catheter irrigation lumen and tube set; to avoid occlusion of the irrigation conduits and prevent ingress of air into the ablation catheter, the ablation catheter may be continuously irrigated when within the vasculature, for example at a rate 2 mL/min; irrigation may only be stopped after removal of the ablation catheter from the body; confirm user selectable ablation parameters on the Generator; plug the ablation catheter with a cable into the RF Generator; observe connector polarity;

Methods herein may include Ablation Catheter Insertion and Ablation Energy Delivery, which may include one or more of the following steps: with the 0.014 guide wire deep seated in the first target intercostal vein, advance the ablation catheter over the guide wire into the intercostal vein; initiate saline tracking (examples of which are set forth herein) from the Generator once the ablation catheter is inserted into the patient; the ablation catheter may be passed from a peripheral vessel to the desired position with the aid of fluoroscopy; the ablation catheter saline infusion rate may be increased to a maximum of 50 mL/min to assist with device entry to the target intercostal vein; place the proximal marker at the anterior midline of the vertebrae in the AP view (if possible); if the azygos to intercostal vein ostium is to the patient's right of midline, advance the device so the proximal radiopaque marker is in the azygos vein proximal to the ostium to the intercostal vein and approximately at the patient's midline; rotate the C-arm to RAO30 (or an appropriate angle that maximizes the projected length between the proximal and distal radiopaque markers) and confirm that the distal marker is not past the costovertebral joint, and adjust as appropriate: confirm that a valid impedance reading (e.g., within 80 to 150 Ohms in monopolar mode, or within 60 to 80 Ohms in bipolar mode) is displayed for both electrodes on the Generator; activate a saline infusion rate of 15 ml/min to 30 ml/min before initiating ablative energy delivery; a recommended saline infusion rate during ablation may be 15 ml/min; The saline infusion rate can be adjusted after initiation of RF delivery to within 15 ml/min to 30 ml/min; initiate the RF ablation mode algorithm from the Generator; monitor the impedance display on the RF Generator, before, during, and after RF power delivery; if a sudden rise in impedance is noted during RF delivery that does not exceed the preset limit, manually discontinue the power delivery; clinically assess the situation; if necessary, remove the ablation catheter and inspect it for damage; in case of a steam pop or automatic shut off, discontinue RF and remove the ablation catheter, terminate saline tracking from the RF Generator and perform a visual inspection, checking for coagulum, charring, or other catheter defects; confirm saline infusion rate and flush the ports prior to reinsertion in the patient, resuming saline tracking once inserted; if the ablation catheter has defects, exchange it for a new one; re-position the ablation catheter and attempt another RF application; in some embodiments, no more than two 180s RF applications should be completed at a single target site; if the pump alarms and stops the irrigation, immediately remove the Catheter from the patient and inspect and re-flush the ablation catheter; when the ablation in the first target intercostal vein (e.g. T11) is finished, remove the guide wire and ablation catheter from the first target intercostal vein and keep in the azygos access sheath in place; the ablation catheter saline infusion rate may be increased to a maximum of 50 cc/min to assist with device removal from the target intercostal vein; the ablation catheter may be removed for inspection; deliver contrast agent to visualize a second target intercostal vein (e.g., T10) from the azygos access sheath; repeat Ablation Catheter Insertion and Ablation Energy Delivery steps to advance the ablation catheter over the guide wire into the second target intercostal vein and ablate: when the ablation in the second target intercostal vein is finished, withdraw the ablation catheter into the 9F azygos access sheath and deliver contrast from the azygos access sheath to obtain a fluoroscopic image of the azygos tree.

Methods herein include device withdrawal, which may include one or more of the following steps: withdraw the ablation catheter into the 9F azygos access sheath and out of the patient; terminate saline tracking; it may be helpful to disconnect the connector cable; inspect the ablation catheter; withdraw the azygos sheath from the patient and close the venous puncture; after use, dispose of the devices in accordance with hospital, administrative, and/or local governmental policy.

In any of the methods herein, including ablation confirmation tests herein, not all of the steps need necessarily to be performed. And some of the steps may occur in different orders. It is of note that the procedures herein are intending to target particular nerves or nerve roots, and are doing so from particular target veins, and even within those veins are placing ablation elements or members within certain regions. The anatomical regions that are being accessed and targeted necessitate certain design requirements. In other treatments that are targeting different anatomical locations for placement, and targeting different target nerves, the device design constraints for those approaches are very different, and thus the devices that can be used in those treatments may be very different. The disclosure herein thus provides specific reasons for designing particular devices, and those reasons include being able to effectively carry out the treatments specifically set forth herein.

While the above description provides examples of one or more processes or apparatuses, it will be appreciated that other processes or apparatuses may be within the scope of the accompanying claims.

### Measuring Central Venous Pressure to Confirm Ablation

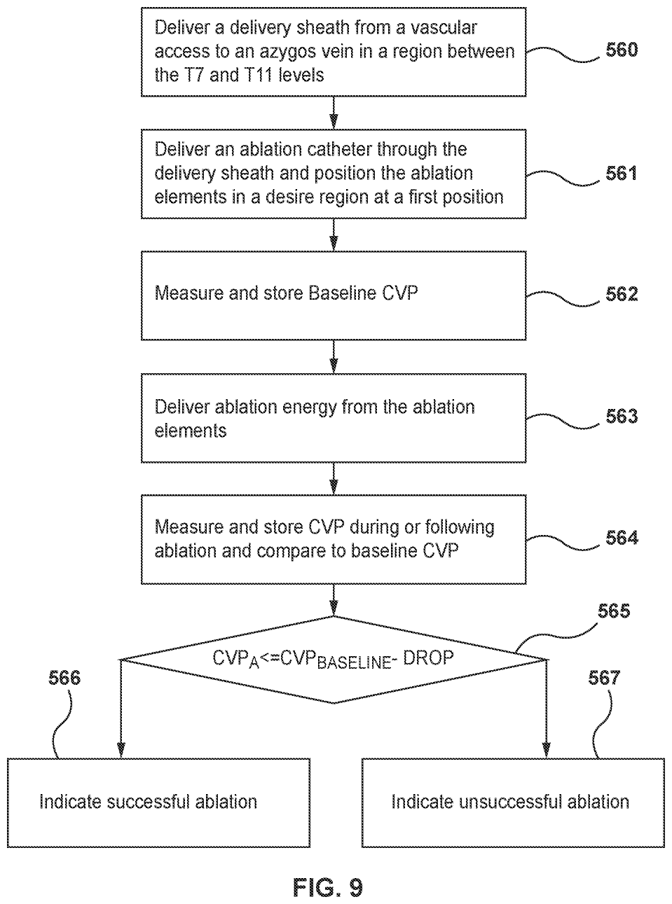

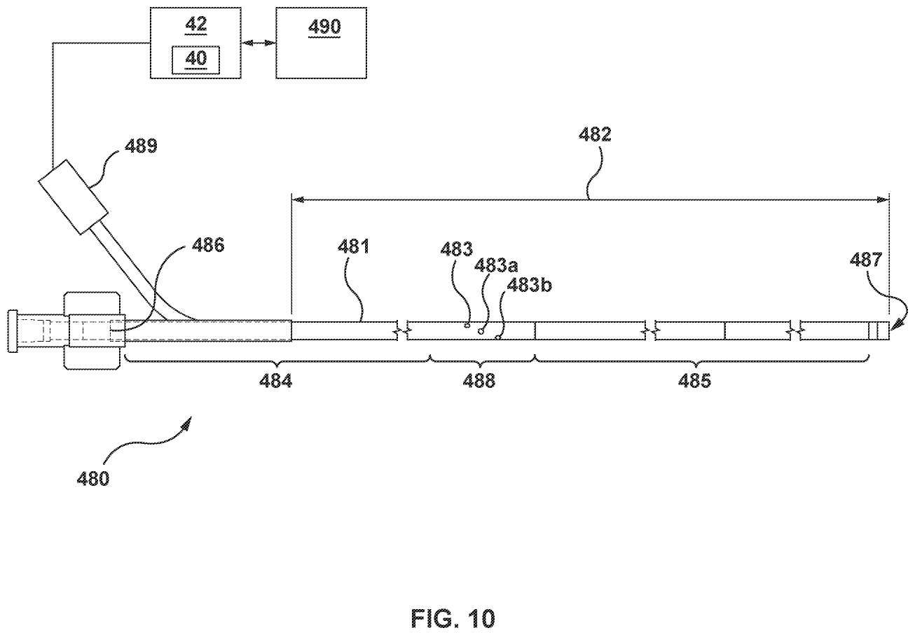

Hemodynamic changes may occur as a result of ablating a GSN. Central venous blood pressure (CVP) may be one of the indicators of GSN ablation. FIG. 9 shows a flow chart with steps for a method of treating a patient by ablating a GSN and using CVP measurements to assess the success of ablation. In the first step 560, a physician may deliver a delivery sheath from a vascular access to an azygos vein in a region between the T7 and T11 levels. The vascular access may be a femoral or jugular vein venotomy. In some embodiments the delivery sheath may have a pressure sensor 483 such as the delivery sheath 505 of FIG. 10. In the second step 561, a physician may deliver an ablation catheter through the delivery sheath and position the ablation elements in a desired region (e.g., in a T9. T10, or T11 intercostal vein) at a first anatomical position. The ablation catheter may be any ablation catheters disclosed herein. In a third step 562, a baseline CVP may be measured and stored immediately (e.g., within 10 minutes, within 5 minutes, withing 1 minute) before ablation energy is delivered. In some embodiments the baseline CVP may be obtained using a pressure sensor on the delivery sheath. In some embodiments the baseline CVP may be assessed and stored by a processor in the ablation control console. In a fourth step 563, ablation energy may be delivered from the ablation elements on the ablation catheter. Ablation energy may be controlled by the ablation control console for example using feedback signals to maintain a setpoint, or using any other control algorithm disclosed herein. In a fifth step 564, CVPA may be measured during (e.g., continuously or discretely) or following GSN ablation and compared to the baseline CVP 565. A predefined drop in CVP (e.g., a drop in more than 10 mmHg, a drop in more than 20 mmHg, a user selected value) may indicate that the GSN was successfully ablated and a user message may be displayed that shows the CVP measurements, the difference between the baseline CVP and second CVP, in some embodiments on a graph with time optionally showing the predefined drop, and/or an interpretation of the CVP measurements (i.e., if CVPA is less than or equal to Baseline CVP-DROP, where DROP=a significant drop (e.g., 10 mmHg. 20 mmHg) then indicate successful ablation of the GSN, 566; an absence of a significant CVP drop may indicate that the GSN was missed or that other splanchnic nerves such as the lesser or least splanchnic nerves or splanchnic nerves on the opposite side require ablation to decrease signal transmission, 567). If the CVP comparison reveals an unsuccessful ablation, a physician may repeat ablation energy delivery at the same level or adjust position and deliver ablation energy in attempt to ablate the GSN at a different level, a different side, or at the same level but different location. The baseline and subsequent CVP measurements for the first ablation may be stored in the console along with an ablation number indicator; while subsequent ablations may include measuring and storing CVP measurements that are stored along with sequential ablation numbers so a user can review the data.

In some embodiments, an electrical stimulation or blocking signal may be delivered to the target nerve (e.g., GSN) while monitoring CVP to assess if the ablation catheter is correctly positioned to ablate the target nerve. The electrical stimulation or blocking signal may be delivered from electrodes on the ablation catheter, for example from the ablation electrodes or from electrodes proximate the ablation electrodes. In one implementation the stimulation electrodes may also function as RO markers, wherein the RO markers are ring bands with one positioned proximal to the ablation electrodes and a second positioned distal to the ablation electrodes, in some embodiments within 2 mm of the ablation electrodes, and the RO markers/electrodes are electrically connected to conductors passing through the catheter to the proximal end of the catheter where they are connectable to a stimulation console. In some embodiments the stimulation console may be incorporated with the ablation console. In some embodiments, during an ablation phase, stimulation signals and ablation signals may be delivered together, for example short bursts of energy may be delivered in a repeating alternating sequence. CVP may be monitored in the patient's inferior vena cava (IVC), in some embodiments with a pressure monitoring delivery sheath.

### Pressure Monitoring Delivery Sheaths

A pressure monitoring delivery sheath may be used to deliver the ablation catheter to the target location and may have a pressure measuring device (e.g., pressure sensor, strain gauge, pressure MEMS) that monitors pressure at a position along the length of the sheath that aligns within the IVC when the distal end of the sheath is in the Azygos vein in a location near the T7 to T11 levels and the access point is a femoral vein venotomy. For example, as shown in FIG. 10, the pressure monitoring delivery sheath 480 may have a tubular section 481 with a lumen and a working length 482 that allows a distal region of the sheath to reach the T7 to T1l levels of an azygos vein from a femoral vein venotomy, wherein the working length 482 of the tubular section 480 is in a range of 50 cm to 115 cm (e.g., about 80 cm), optionally wherein the working length is 70 cm to 115 cm when the access point is a femoral vein and 50 cm to 85 cm when the access point is a jugular vein. When placed in a large patient the proximal 20 cm or so may be positioned in the femoral vein and the distal 24 cm or so may be in the azygos vein. In a smaller patient some of the proximal end may be outside the patient and about 12 cm may be in the azygos vein. A pressure sensor 483 may be positioned on or in a wall of the tubular section 481 in a range 488 that is in a range 484 of 32 cm to 56 cm from the proximal end 486 of the tubular section so that the sensor 483 is located in the vena cava in most patients when the delivery sheath is positioned for delivery of an ablation catheter. Alternatively stated, a pressure sensor 483 may be disposed in a range 488 that starts 32 cm to 56 cm from a proximal end 486 of the tubular section. A pressure sensor may be positioned on the delivery sheath in a range 488 that is in a range 485 of 24 cm to 48 cm from the distal end 487 of the tubular section 481 so that the sensor 483 is located in the vena cava in most patients when the delivery sheath is positioned for delivery of an ablation catheter. Alternatively stated, a pressure sensor 483 may be disposed in a range 488 that starts 24 cm to 48 cm from a distal end 487 of the tubular section.

An alternative delivery approach may include accessing a jugular vein and delivering a delivery sheath from the jugular venotomy to the azygos vein at the T7-T11 level. A delivery sheath for jugular access may be shorter than some sheaths described herein (e.g., 50 cm to 85 cm) but the position of the pressure sensor on or in a wall of the tubular section may be in a similar range 485 from the distal end 487 (e.g., in a range of 24 cm to 48 cm from the distal end).

Pressure sensors herein may include one or more types of pressure sensors such as optical, strain, film, variable capacitance or other form of small medical grade sensor, which may be in the form of a Micro Electro-Mechanical System (MEMS) sensor. For example, any of the pressure sensors herein may be positioned in the wall of the delivery sheath with a pressure-transmitting protective cover, such as a flexible membrane or sealant covering the sensor to protect the sensor as well as provide a smooth surface on the sheath.

FIG. 10 illustrates an example in which the sheath optionally includes a plurality of pressure sensors. A plurality of pressure sensors 483, 483a, 483b may be placed or disposed on the delivery sheath on different radial or circumferential sides and different axial positions in the range 488 of the tubular section so that if one sensor is pressed against a vessel wall another sensor may be unimpeded so it can be used to read CVP. The sensor(s) 483, 483a, 483b may be electrically connectable via a connector 489 to a pressure monitoring console 490, which may be incorporated with the ablation console 42. In some embodiments, the ablation energy delivery algorithm 40) may accept inputs from the pressure sensor 483 or pressure console 490 to automatically control the ablation energy delivery and/or electrical stimulation or blocking energy.

Delivery Sheaths with Deployable Balloon

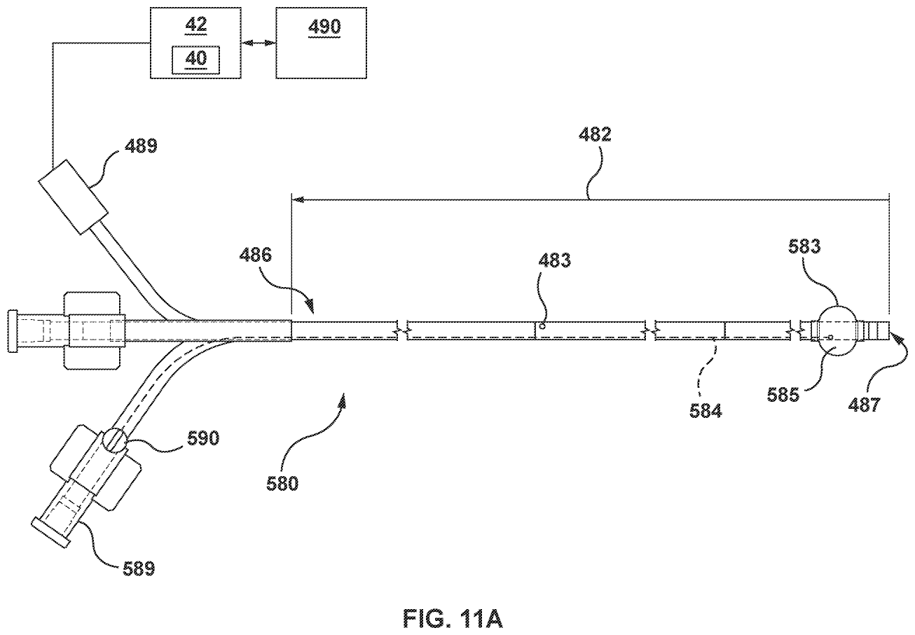

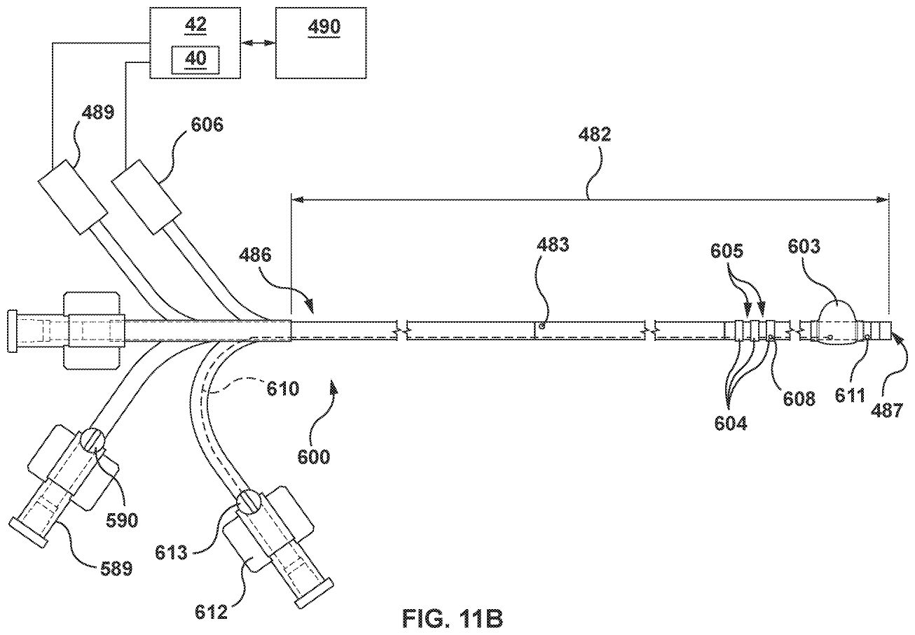

Any of the delivery sheaths herein may further include an inflatable structure proximate a distal end of the tubular section of the sheath. It is understood, however, that delivery sheaths herein that include an optional deployable structure need not include features of other sheaths herein (e.g., one or more pressure sensors). An exemplary delivery sheath 580 may, in some embodiments, have a deployable balloon 583 on or near its distal end 487, deployable from the outer surface, as shown in FIG. 11A (distal end 487 may also be the distal end of a tubular section of the sheath, wherein exemplary details of a tubular section of the sheath are described with respect to FIG. 10). Alternatively, a deployable balloon may be on the outer surface of the distal end of a dilator that is configured to pass through the delivery sheath. The balloon 583 may be deployed to facilitate delivery of the sheath 580 over the arch of the azygos vein, for example to adjust stiffness of the sheath or to redirect the tip's trajectory to traverse a veinous valve. The balloon 583 may be used to facilitate radiographic visualization of the vasculature in the target region. Since blood flows in the azygos vein retrograde to the direction of delivery (i.e., towards the head), injecting contrast into the vasculature (e.g., azygos vein) from the delivery sheath 580 may flow preferentially backwards instead of into the intercostal veins where it is desired to go. Occluding (e.g., fully or partially) the azygos vein before injecting contrast solution may facilitate delivery of contrast into the intercostal veins where it may dwell for a longer duration, which can help a physician visualize the position of the target vessel and other landmarks such as the ostium from the azygos to intercostal vein, or tortuosity of the vessels. The balloon 583 may be made from a compliant balloon material and may have a lubricious coating on the outer surface. The deployable balloon 583 may be deployed during delivery or removal of the ablation catheter (e.g., any of the ablation catheters disclosed herein) to help stabilize the delivery sheath so forces applied to the ablation catheter handle or shaft are transferred more easily to the distal region of the catheter. The deployable balloon 583 may be radially symmetric around the delivery sheath 580. A balloon-inflation-lumen 584 positioned in the wall of the delivery sheath 580 and in fluid communication with the inner space 585 of the deployable balloon 583 may be used to inflate or deflate the balloon 583. The lumen may be in fluid communication with a connector 589 on the proximal end of the sheath that is connectable to a fluid delivery device such as a syringe or pump. The connector 589 may have a valve 590, that when closed seals air flow in the lumen and maintains air pressure in the balloon 583.

In some embodiments, a deployable balloon 603 may be radially asymmetric or positioned preferentially on one side of a delivery sheath 600, and the delivery sheath may further have a return electrode 604 on at least the opposite side of the balloon 603, on the outer surface of the delivery sheath 600, and within 15 cm (e.g., within 5 cm) of the distal end 487 of the delivery sheath. The electrode 604 may be a dispersive electrode with a larger surface area than the ablation electrodes that is electrically connected to a connector 606 that connects to a console 42 via a connector cable, and that completes an electrical circuit with the one or more ablation electrodes on an RF ablation catheter through the console 42, thus eliminating the need for an external grounding electrode. In some embodiments, a return electrode 604 on the delivery sheath, may be used to complete a circuit with stimulation electrodes. The return electrode may have a surface area in a range of 10 mm² to 200 mm² (e.g., in a range of 80 mm² to 150 mm²). In some embodiments the return electrode 604 may include a plurality of band electrodes each having a length in a range of 1 mm to 10 mm and spaced from one another (e.g., with a space 605 in a range of 5 to 10 mm) so the delivery sheath remains sufficiently flexible. The return electrode 604 may be made with a radiopaque material. A temperature sensor 608 may be positioned in the wall of a delivery sheath, in some embodiments on the opposite side of an asymmetric deployable balloon 603 so it can measure temperature of the wall of the azygos vein or blood flowing past the sensor. The temperature sensor 608 may be electrically connected to a connector 606, which is configured to send a temperature signal to the console 42. The temperature signal from the delivery sheath may be used in assessment of safety wherein a warning message is displayed or a reaction in the ablation control algorithm is made such as reduction of power.

Radiopaque contrast solution may be injected through a delivery sheath, for example through the sheath's central lumen which may also be used to slidably engage a guidewire, diagnostic catheter, or ablation catheter. In some embodiments as shown in FIG. 11B, the delivery sheath 600 may have a lumen 610 in its sidewall for delivery of contrast. In one implementation the contrast-delivery-lumen 610 may be the same lumen as the balloon-inflation-lumen. In another implementation, a sheath may have a deployable balloon with a balloon-inflation-lumen and a separate contrast delivery lumen 610. In some embodiments, the contrast delivery lumen may terminate in a port 611 at or near the distal end of the delivery sheath with a pressure release valve that opens upon pressurization of the contrast delivery lumen, for example above a release pressure in a range of 50 to 150 mmHg, or when the differential pressure between the lumen and exterior of the valve is in a range of 50 to 150 mmHg. In embodiments wherein the contrast delivery lumen 610 is separate from a balloon inflation lumen 584, the contrast delivery lumen 610 may be in fluid communication with a Luer connector 612 and valve 613, which may be visually distinguishable from the balloon inflation connector 589.

A delivery sheath in some embodiments may have a predefined curved tip, which may facilitate traversing the azygos arch or a veinous valve. Alternatively, a dilator configured for use with the delivery sheath, may have a predefined curved tip. The curved tip may be radiopaque or have a radiopaque element. In some embodiments, the dilator may have features that allow it to be left in place in the patient's vasculature while a delivery sheath is removed proximally from the dilator and in some embodiments other sheaths or catheters may be delivered over the dilator. The dilator may have a length that is at least twice as long as the delivery sheath, for example in a range of 200 cm to 700 cm long. The proximal end of the dilator may have a narrow profile, smaller than the ID of the delivery sheath, so the sheath may be removed proximally off of the dilator. A dilator may have a proximal hub that is removable, for example tear-away, which is removed if desired. A dilator may have a proximal hub that is compressible.

Delivery Systems with a Delivery Sheath and Two Dilators

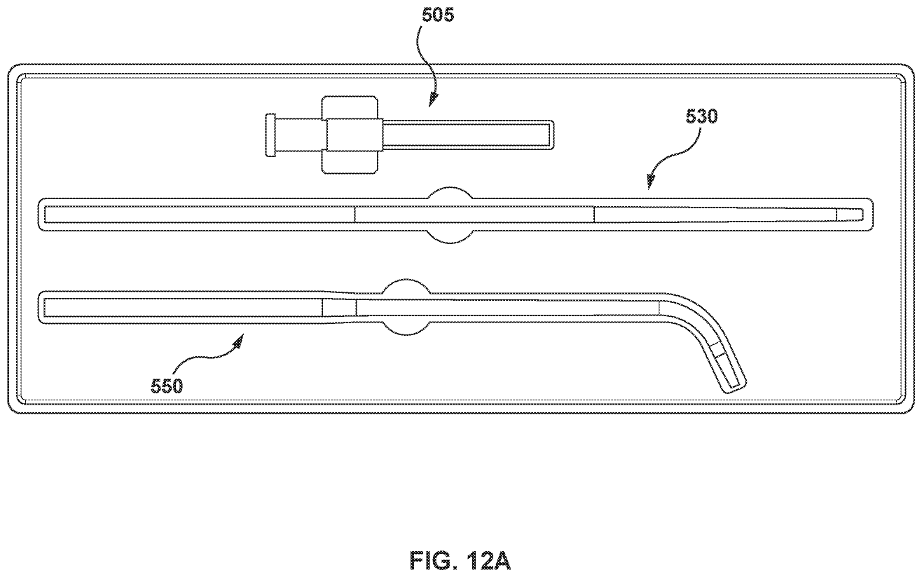

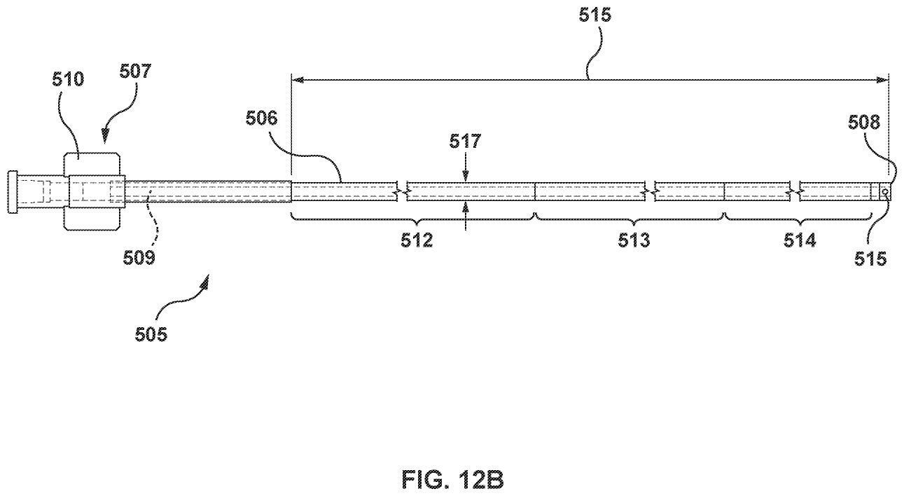

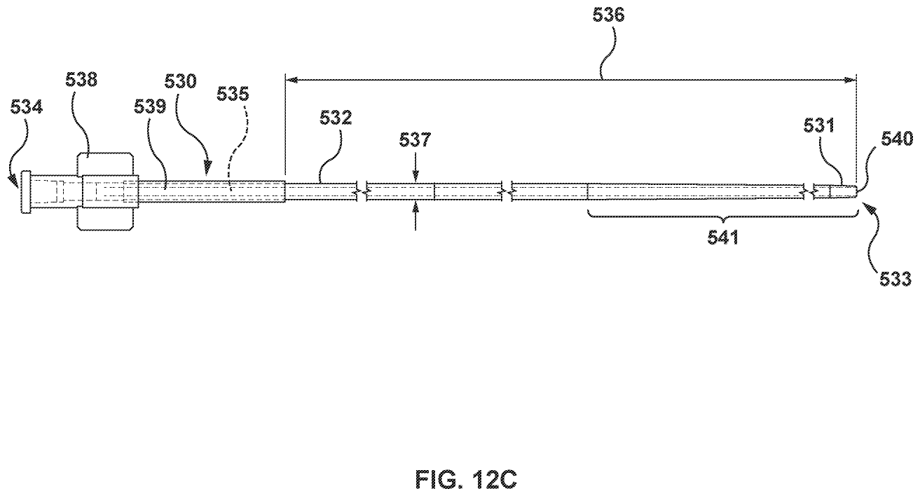

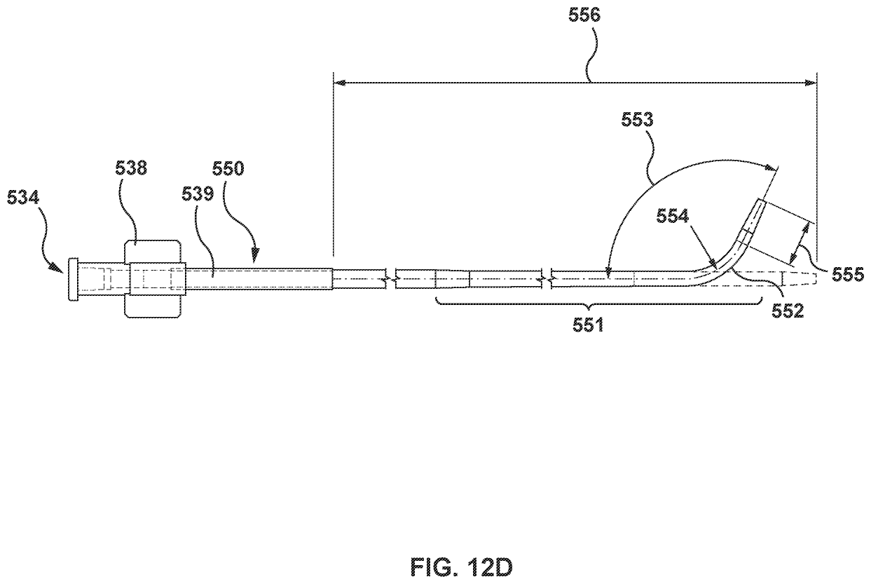

A GSN ablation catheter delivery system 500 may include a delivery sheath 505, a first dilator 530 and a second dilator 550, as shown in FIG. 12A, and may be provided as a kit. The delivery sheath 505 may be any of the delivery sheaths described herein, and any details related to sheath 505 may be incorporated into any of the sheaths herein. The delivery sheath 505 may have an elongated tubular structure 506 with a proximal end 507 and a distal end 508 and a lumen 509 therebetween. The lumen 509 may have an inner diameter configured to slidably contain the ablation catheter (e.g., any of the ablation catheters disclosed herein), for example a 9F compatible inner diameter (about 3.35 mm). The elongated tubular structure 506 may have an outer diameter 517 of about 12F. The proximal end 507 of the tubular structure 506 may be connected to a handle or connector 510, such as a female Luer or a handle with a hemostasis valve. The tubular structure 506 may have an inner liner of PTFE. The tubular structure 506 may be reinforced with a braided wire layer 511 embedded in polymer. The tubular structure 506 may have a higher stiffness at the proximal end 507 and a lower stiffness at the distal end 508. For example, the difference in stiffness may be accomplished by adjusting the braid density (e.g., from a proximal braid density of 80 PPI to a distal braid density of 40 PPI), and/or polymer durometer. The difference in stiffness may be gradual along the length of the tubular structure or change in sections. For example, the tubular structure 506 may have a proximal section 512 with a first stiffness, a middle section 513 with a second stiffness, and a distal section 514 with a third stiffness, wherein the third stiffness is less than the first stiffness and the second stiffness is between that of the first and third stiffness. An example construction of the tubular structure 506 with varying stiffness sections may have a proximal section 512 with a braid density of 80 PPI and a polymer jacket made from Pebax™ 7233 (e.g., having a durometer of 72D), a middle section 513 with a braid density of 60 PPI and a polymer jacket made from Pebax™ 6333 (e.g., having a durometer of 63D), and the distal section 514 with a braid density of 40 PPI and a polymer jacket made from Pebax™ 5533 (e.g., having a durometer of 55D). Each section may have an equal inner diameter (e.g., 3.35 mm) and wall thickness (e.g., 0.127 mm). The total working length 515 of the tubular structure 506 may be sufficient, in most patients, to reach the T11 level in an azygos vein from an access point in a vein such as a femoral vein or jugular vein when passing through vasculature such as from the access vein to a vena cava to an azygos vein, which may be in a range of 50 cm to 115 cm (e.g., about 80 cm+/−0.5 cm). In some examples where the access point is a jugular vein, working length 515 may be 50 cm to 85 cm. In some examples where the access point is a femoral vein, working length 515 may be 70 cm to 115 cm. The sections 512, 512, 514 may be fabricated separately and connected (e.g., welded), made as one piece, or a combination thereof. The distal section 514 may have a length of 9.50+/−0.50 cm. The middle section 513 may have a length of 6.5+/−0.5 cm. The proximal section 512 may have a length that is the remainder of the total working length 515, for example about 64 cm. The variable stiffness of the sheath 505, in some embodiments along with use of the first or second dilators contained in the sheath, and in some embodiments along with the use of a guidewire in a dilator or in the sheath without a dilator, may provide the various functions that facilitate delivery of an ablation catheter from a vena cava, over an azygos vein arch, and descending down the azygos vein to a level around the T7 to T11 intercostal veins. The proximal section 512 may function to transmit translation and rotation forces applied to the proximal end 507 (e.g., female Luer 510) through the proximal section 512 to the distal section 514, yet permit bending in order to traverse anatomical bends in the vasculature. The less stiff distal section 514 may facilitate getting the sheath to enter the azygos vein from the vena cava. In particular, when the first dilator 530) or second dilator 550 is advanced over a guidewire from within the delivery sheath 505 positioned in a vena cava, and through an azygos vein arch and in some embodiments down the azygos vein to a level in the T7 to T11 region, the delivery sheath 505 may be advanced over the dilator 530 or 550 and the flexible distal section 514 will be flexible enough to conform and follow over the bend of the dilator, without getting stuck or causing the dilator to get pulled out of position. The middle section 513 functions as a flexibility transition to prevent kinking, which is more likely to happen with sections having a greater change in flexibility. The distal end 508 of the delivery sheath may have a soft, atraumatic tip 515 to protect vessel walls from injury, and may have an integrated radiopaque marker such as a platinum iridium marker band.

An exemplary first dilator 530 is shown in FIG. 12C. An exemplary second dilator 550 is shown in FIG. 12D and uses the same callout numbers for features that are the same as in the first dilator. The first and second dilators 530, 550 may have different features and handling characteristics that, along with the delivery sheath 505, facilitate entering the azygos vein from the vena cava, traversing the azygos vein arch, reaching to the T7 to T11 level of the azygos vein, delivering contrast agent to a portion of the azygos and intercostal veins, delivering a guide wire (e.g., 0.035″ guidewire) into a desired intercostal vein to prepare for the delivery of the ablation catheter to the desired intercostal vein (e.g., a T9. T10, or T11 intercostal vein). The dilators may further function to dilate a perforation such as a venotomy to accept the delivery sheath. To accomplish these functions the dilators 530 and 550 may each have a tapered distal tip 531, at the distal end 533 of an elongated tubular structure 532, wherein the elongated tubular structure 532 may have a distally decreasing outer diameter, and a lumen 535 with a consistent or uniform inner diameter. The dilators may have a working length 536 that allows the elongated tubular structure 532 to extend beyond the distal end 508 of the delivery sheath 505 more than traditional dilators, for example by an amount in a range of 10 to 30 cm (e.g., about 18 cm), which allows the dilator to be advanced into vasculature ahead of the delivery sheath by up to the extended length, and then the sheath 505 to follow over the dilator 530, 550. As such, the dilator(s) can be used to guide the delivery sheath 505 through tortuous vasculature and the extended length allows the dilator to be “deep seated”, in other words advanced well beyond the delivery sheath so that when the delivery sheath is advanced over the deep seated dilator it doesn't cause the dilator to be pulled out of place and the dilator then provides a track for the sheath to follow over to traverse tortuous vessels. The two dilators 530, 550 may each be suitable for different specific steps in the delivery procedure or they may provide different handling characteristic to give the user more options when encountering challenges in accessing the desired position in the azygos vein. The first and second dilators 530, 550 may have an elongated tubular structure 532 with a maximum outer diameter (e.g., OD of the proximal section) 537 that slidably fits into the lumen 509 of the delivery sheath 505, a lumen 535 between the proximal 534 and distal 533 ends with an inner diameter configured to slidably contain a 0.035″ guidewire, a female Luer 538 connected to the proximal end of the elongated tubular structure 532 in some embodiments with a strain relief 539 therebetween, and a distal tip 531 that is tapered with a distal edge 540 that is rounded. The first dilator 530 may have a distal section 541 that extends from the distal end of the delivery sheath 505 and has a stiffness that is less than the distal section 514 of the delivery sheath. The first dilator's distal section 541 may have a stiffness that decreases toward the distal end. The stiffness may be varied by tapering or stepping down the outer diameter or wall thickness or by varying materials or arrangement of materials such as with a braided wire having varying braid density. The first dilator may have a total working length 536 in a range of 60 cm to 145 cm, optionally 107+/−0.5 cm, and a distal tapered section 541 having a length in a range of 3 to 10 cm, preferably 5 cm+/−0.5 cm, and a tapered distal tip 531 having a length in a range of 3 to 10 mm, preferably 5 mm+/−0.5 mm. The second dilator 550 may have a distal section 551 that is different than the distal section 541 of the first dilator, and that extends from the distal end of the delivery sheath 505 and has a stiffness that is less than the distal section 514 of the delivery sheath and less than the distal section 541 of the first dilator 530. The second dilator may have a total working length 556 in a range of 60 cm to 145 cm, optionally 107+/−0.5 cm, and a distal tapered section 551 having a length in a range of 5 to 15 cm, preferably 9 cm+/−0.5 cm, and a tapered distal tip 531 having a length in a range of 3 to 10 mm, preferably 5 mm+/−0.5 mm. Furthermore, the second dilator 550 may have a preformed curve 552 on its distal section 551, wherein the preformed curve in its unconstrained state (e.g., as seen without being constrained in a delivery sheath or having a guidewire in its lumen), as shown in FIG. 12D may have an angle 553 in a range of 90 degrees to 120 degrees (e.g., about 115 degrees), a radius of curvature 554 in a range of 7 to 11 mm (e.g., about 9.14 mm) and a straight section 555 distal to the preformed curve with a length in a range of 5 mm to 10 mm (e.g., about 7 mm). The extended length of the dilators that extends from the delivery sheath 505 allows the dilator(s) to be advanced well into the azygos vein (e.g. by an amount up to the length that extends beyond the delivery sheath, or to the level of azygos vein between the T7 and T11 vertebra) before advancing the delivery sheath 505, which may provide sufficient structural support to allow the delivery sheath to follow over the dilator without causing the dilator to fall out of position.

The second dilator 550) has the same features as the first dilator 530 except the distal section may be longer, more flexible, and have a preformed curve. The preformed curved tip can be used to initially access an anatomical feature, such as an ostium leading into the azygos vein from the vena cava. The curved tip of the dilator may be rotationally positioned by applying torque at the proximal end of the dilator. The construction of the dilator can be a simple extruded tube or alternatively may have a composite construction with wall reinforcement, such as wire braid and polymer. An alternate construction combines a distal extruded tube connected to a proximal composite tube. The curved tip of the dilator can have such flexibility that a flexible (floppy) distal portion of a guidewire positioned in its lumen may leave the curved tip unchanged while a stiffer more proximal section of the guidewire may straighten the curved tip. This may promote the continued use of the dilator with the delivery sheath after its initial purpose to access the anatomical feature. i.e., ostium, where when straightened may more easily translate along a vein passage. The second dilator 550 may have a single bend 552 or have multiple bends. The preformed curve 552 may allow a guidewire to exit up to 90 degrees relative to the long axis of the dilator 550. The very distal tip of the dilator may be atraumatic, for example having a hemispherical or bullet shape, to prevent injuring the vessel wall.

The delivery sheath, first dilator and second dilator may be packaged together as a kit in a sterile package, which may further contain a guidewire.

An ablation catheter may be similar to any of the ablation catheters shown in FIGS. 5A to 5E and in some embodiments have additional coil electrodes, for example a total of three coil electrodes each in some embodiments with a length in a range of 5 mm to 10 mm. Features described in relation to the implementations shown in FIGS. 5A to 5E may in some embodiments be incorporated into a catheter having three or more ablation electrodes. A three-electrode ablation catheter may be useable in a wide range of patients wherein a one, two, or three electrode ablation may be chosen depending on the length of a desired ablation. Some ablation procedures may require a two-electrode ablation, wherein the third electrode is inactive, for example if the distance within a target intercostal vein between the azygos ostium and costovertebral joint or sympathetic trunk is within a range of 18 mm to 25 mm, the azygos is right-biased, or the angle of the intercostal vein is transverse to the spinal column plus or minus about 10 degrees. In some situations a longer ablation may be required to ensure the GSN is ablated and the third ablation electrode may be activated in addition to the first and second electrodes, for example if the distance within a target intercostal vein between the azygos ostium and costovertebral joint or sympathetic trunk is within a range of 20 mm to 35 mm, the azygos is left-biased, or the angle of the intercostal vein is more than 10 degrees from transverse to the spinal column (e.g., more than 15 degrees, more than 20 degrees, more than 25 degrees, more than 30 degrees). The three or more electrodes may be radiopaque or be associated with radiopaque markers and a user may determine of the proximal electrode is within the intercostal vein or in the azygos vein when the distal radiopaque marker is positioned at or near the costovertebral joint and if so the proximal electrode may be deactivated by instructing the console to do so. Alternatively, a console may automatically select or deselect one or more of the ablation electrodes by assessing a sensor associated with the electrodes such as an impedance or thermal sensor.

In some embodiments, a method of use may include placing a thermal sensing catheter in the patient's esophagus in close proximity to the ablation site to monitor temperature of the esophagus, for example the inner surface of the esophagus. If temperature monitored in the esophagus increases, for example by 1 degree Celsius above body temperature, a warning may be given or the temperature signal may be delivered to the ablation console and a control algorithm may decrease power or set temperature or stop energy delivery.

To the extent any amendments, characterizations, or other assertions previously made (in this or in any related patent applications or patents, including any parent, sibling, or child) with respect to any art, prior or otherwise, could be construed as a disclaimer of any subject matter supported by the present disclosure of this application, Applicant hereby rescinds and retracts such disclaimer. Applicant also respectfully submits that any prior art previously considered in any related patent applications or patents, including any parent, sibling, or child, may need to be re-visited.

Specific embodiments described herein are not intended to limit any claim and any claim may cover processes or apparatuses that differ from those described below, unless specifically indicated otherwise. The claims are not limited to apparatuses or processes having all of the features of any one apparatus or process described below or to features common to multiple or all of the apparatuses described below, unless specifically indicated otherwise. It is possible that an apparatus or process described below is not an embodiment of any exclusive right granted by issuance of this patent application. Any subject matter described below and for which an exclusive right is not granted by issuance of this patent application may be the subject matter of another protective instrument, for example, a continuing patent application, and the applicants, inventors or owners do not intend to abandon, disclaim or dedicate to the public any such subject matter by its disclosure in this document.

1. A catheter delivery system (500), comprising:
a delivery sheath, a first dilator (530) and a second dilator (550), wherein the first and second dilators each comprise a dilator distal section (541, 551) that extends from a distal end (508) of the delivery sheath (505) when fully inserted by an extension amount in a range of 10 cm to 30 cm.

2. The catheter delivery system of claim 1, wherein each of the dilator distal sections (541, 551) comprises a stiffness that is less than a stiffness of a distal section (514) of the delivery sheath.

3. The catheter delivery system of claim 2, wherein the stiffness of the second dilator distal section (551) is less than the stiffness of the first dilator distal section (541).

4. The catheter delivery system of claim 2 or claim 3, wherein the stiffness of the dilator distal sections (541,551) decreases in a distal direction.

5. The catheter delivery system of claim 4, wherein the dilator distal sections comprise a distally decreasing outer diameter.

6. The catheter delivery system of claim 5, wherein the distally decreasing outer diameter comprises a gradual taper, with stepped down outer diameters, or with a combination thereof.

7. The catheter delivery system of any one of claims 1 to 6, wherein the first dilator and the second dilator each comprise a dilator tubular structure, a proximal end, a distal end, a working length between the proximal and distal ends, a central lumen therebetween, a distal section with a distally decreasing outer diameter, and a tapered distal tip.

8. The catheter delivery system of claim 7, wherein each of the working lengths (536, 556) is in a range of 60 cm to 145 cm.

9. The catheter delivery system of claim 7 or claim 8, wherein the first dilator distal section (541) has a length in a range of 3 to 10 cm, preferably 5+/−0.5 cm.

10. The catheter delivery system of any one of claims 7 to 9, wherein the second dilator distal section (551) has a length in a range of 3 to 10 cm, preferably 5+/−0.5 cm.

11. The catheter delivery system of any one of claims 7 to 10, wherein the second dilator comprises a preformed curve (552) on a distal section (551).

12. The catheter delivery system of claim 11, wherein the preformed curve (552), when in an unconstrained state, comprises an angle (553) in a range of 90 degrees to 120 degrees, preferably 115 degrees, a radius of curvature 554 in a range of 7 to 11 mm, preferably 9.14 mm.

13. The catheter delivery system of claim 11 or claim 12, wherein the second dilator comprises a straight section (555) distal to the preformed curve with a length in a range of 5 mm to 10 mm, preferably 7 mm.

14. The catheter delivery system of any one of claims 7 to 13, wherein the tapered distal tip of each dilator has a length in a range of 3 to 10 mm, preferably 5+/−0.5 mm.

15. The catheter delivery system of any one of claims 1 to 14, further comprising a guidewire.

16. The catheter delivery system of any one of claims 1 to 15, wherein the delivery sheath comprises a proximal end and a distal end, a lumen therebetween, and a tubular structure (506) comprising a braided wire and a polymer.

17. The catheter delivery system (500) of claim 16, wherein the tubular structure has a variable stiffness that decreases towards the distal end.

18. The catheter delivery system (500) of claim 17, wherein the variable stiffness changes on a graduation.

19. The catheter delivery system (500) of claim 17, wherein the variable stiffness changes in sections.

20. The catheter delivery system (500) of any one of claims 17 to 19, wherein the variable stiffness is created by varying a braid density of the braided wire.

21. The catheter delivery system (500) of claim 20, wherein the braid density proximate the proximal end has 80 PPI, and the braid density proximate the distal end has 40 PPI.

22. The catheter delivery system (500) of claim 17, wherein the tubular structure comprises a proximal section comprising a first stiffness, a middle section comprising a second stiffness, and a distal section comprising a third stiffness, wherein the third stiffness is less than the first stiffness and the second stiffness is between that of the first and third stiffnesses.

23. The catheter delivery system of claim 22, wherein the proximal section comprises a braid density of 80 PPI and a polymer with a durometer of 72D, the middle section comprises a braid density of 60 PPI and a polymer with a durometer of 63D, and the distal section comprises a braid density of 40 PPI and a polymer with a durometer of 55D.

24. The catheter delivery system of claim 22 or claim 23, wherein the proximal, middle, and distal sections each comprise an inner diameter equal to one another.

25. The catheter delivery system of claim 24, wherein the inner diameter is 3.35 mm.

26. The catheter delivery system of any one of claims 22 to 25, wherein the proximal, middle, and distal sections each comprise a wall thickness equal to one another.

27. The catheter delivery system of claim 26, wherein the wall thickness is 0.127 mm.

28. The catheter delivery system of any one of claims 1 to 27, wherein the system is configured for delivering an ablation catheter from a vasculature access point to the patient's azygos vein at a level between T7 and T11.

29. The catheter delivery system of any one of claims 1 to 28, wherein the system is configured for delivering an ablation catheter from a vasculature access point to the patient's intercostal vein a level between T7 and T11.

30. The catheter delivery system of claim 28 or 29, wherein the working length (515) of the tubular structure of the sheath is in a range of 50 cm to 115 cm.

31. The catheter delivery system of claim 30, wherein a distal section of the sheath comprises a length of 9.50+/−0.50 cm.

32. The catheter delivery system of claim 30 or claim 31, wherein a middle section of the sheath comprises a length of 6.5+/−0.5 cm.

33. The catheter delivery system of any one of claims 30 to 32, wherein a proximal section of the sheath has a length that is the remainder of the working length minus a length of the distal section and a length of the middle section.

34. The catheter delivery system of claim 33, wherein the proximal section of the sheath has a length of 64 cm.

35. The catheter delivery system of any one of claims 1 to 34, wherein the delivery sheath is any delivery sheath herein.

36. The catheter delivery system of any one of claims 1-35, wherein the catheter delivery system is provided as a kit in a sterilized package.

37. A method of using a delivery system, comprising:
advancing a delivery sheath within a patient;
advancing a dilator from with the delivery sheath beyond a distal end of the delivery sheath;
advancing the dilator into an azygos vein from a vena cava; and
further advancing the delivery sheath over the dilator and into the azygos vein.

38. The method of claim 37, wherein advancing the dilator beyond a distal end of the delivery sheath comprises advancing the dilator from 10 cm to 30 cm beyond a distal end of the delivery sheath.
